(19) United States
(12) Patent Application Publication
(10) Pub. No.: US 2026/0357182 A1
(43) Pub. Date: Nov. 12, 2026

(54) FLUID TRANSFER SYSTEM, APPARATUS AND METHODS FOR BIOPROCESSING

(71) Applicant: CELLULAR ORIGINS LIMITED, Melbourn (GB)

(72) Inventors: Dan Strange, Melbourn (GB); Leo Steenson, Melbourn (GB); Ryan Roberts, Melbourn (GB); Peter Crossley, Melbourn (GB); Paul Crisp, Melbourn (GB); Ed Stone, Melbourn (GB); William Deeks, Melbourn (GB)

(21) Appl. No.: 19/107,343

(22) PCT Filed: Aug. 31, 2023
(86) PCT No.: PCT/GB2023/052262

§ 371 Date: Feb. 27, 2025

(30) Foreign Application Priority Data
Aug. 31, 2022 (GB) 2212684.1
Aug. 31, 2022 (GB) 2212685.8
May 26, 2023 (GB) 2307961.9
May 26, 2023 (GB) 2307962.7
May 26, 2023 (GB) 2307963.5

(51) Int. Cl. C12M 1/26; C12M 1/00; F16L 13/02

(52) U.S. Cl. CPC C12M 33/00; C12M 23/26; C12M 29/00; C12M 29/04; F16L 13/02

(57) ABSTRACT

A device for collecting a fluid sample from a fluid-containing consumable in a bioprocessing system, the consumable having a tube fluidly attached thereto, the device comprising: a fluid connection portion configured to be connected to the tube of a fluid-containing consumable, and to enable pumping of fluid or air through the device; and an air input portion configured to allow sterile air to be supplied to the device; a sampling portion connected to the fluid connection portion, wherein during pumping into the device, the sampling portion is configured to receive a fluid sample from the consumable via the fluid connection portion; a bypass tube having a first end connected to the fluid connection portion and a second end connected to the air input portion, wherein the bypass tube is configured to receive air supplied from the air input portion and direct the air out of the device via the fluid connection portion without passing through the sampling portion, whereby to purge fluid from the tube connected to the consumable; and means for restricting fluid flow through each of the sampling portion and bypass tube, such that fluid being pumped into the fluid connection portion passes only into the sampling portion and air pumped out of the fluid connection portion passes only through the bypass tube.

### FIELD OF THE INVENTION

The present invention relates to automated bioprocessing systems, such as bioprocessing systems for performing cell therapy.

### BACKGROUND

Therapeutics are increasingly using cells rather than small molecules as the starting point. The approaches to manufacturing these products are rapidly evolving to keep up with constantly emerging new therapies. In recent years, there has been an increased use of a number of new classes of cell therapies. One class is autologous cell therapies.

Autologous cell therapies are a promising class of therapy, which have significant clinical and commercial potential ranging from treating cancer to fixing genetic defects. These therapies involve taking cells from a patient, manipulating the cells over the course of days to weeks, and re-introducing the cells back into that patient's body to produce a therapeutic effect. The steps taken during autologous cell therapies are often complex; for example, a typical CAR-T process may involve a sequence of steps starting with a cryopreserved leukopak, thawing, washing to remove DMSO, enrichment of T cells, activation, transduction, expansion, concentration, formulation fill finish into an IV bag, and cryopreservation, with several other intermediate washing steps.

Bioprocessing systems have been developed for carrying out the above steps. Within such a bioprocessing system, multiple different consumables (e.g. fluid-containing bags) may be required to supply media, reagents and/or cell material to a separate consumable known as an “expansion chamber” (or “cell culture chamber”), for example, throughout the cell therapy process. Similarly, one or more consumables (e.g. an output or waste bag consumable) may receive fluid samples from the expansion chamber. The term “consumable” may therefore be used to refer to a “single-use” element or component of the system.

Some of the processes discussed above such as activation, transduction and expansion may be carried out within a cell culture chamber (or “bioreactor”), which may be incubated. During one or more of those processes, various functions need to be performed, such as media addition and removal, harvesting and sampling.

However, due to the need to maintain a closed system when handling the cells, reagents and other fluids, conventional consumables (e.g. media bags) include a complicated network of external flexible tubing for interfacing with the incubation chamber. This network of flexible tubing can be difficult to handle, particularly by an automated means such as a robotic device.

An alternative approach is to connect each consumable to its own flexible tube and perform the required connections individually between the consumables throughout the process. More specifically, tube welding may be used to attach flexible tubes together, a pump may transfer fluid through the connected tubes, and the tubes may be disconnected again to facilitate further connections being made between other consumables.

However, there are a large number of reasons that would make tube welding appear incompatible with full (operator independent) automation. For example, tube welding requires the handling of long flexible tubes, which are well known to be extremely challenging to handle by automated handling means, such as robots. For example, flexible tubing is unlikely to stay within a well-defined deterministic location when moved by a robot, making engagement, alignment and orientation of the tubes challenging.

When the end of a sufficiently long flexible tube, constrained at one end, is manipulated at the other, the path defined by the tube will have a number of non-unique solutions as to what the path between the constrained end and the manipulated end will take-up, dependent on the internal stress in the tube. Such a system has many degrees of freedom. Such applications are ideal for humans but very challenging for automation and therefore typically left for human operators. Since weld strength is very dependent on precise positioning and compression of the tube, any errors in engagement of the tubing by a robot may lead to failure of the weld. It is also challenging to avoid tubes becoming twisted and/or entangled with other tubes and with other parts of the bioprocessing system. Furthermore, the tubes may apply unexpected tension to the robot or any attached consumables, reducing the chance of success of the weld, or potentially damaging the connection to the consumable.

A further problem that may occur when using lengths of flexible tubing, is that when fluid is pumped between a first consumable and a second consumable, even when sufficient fluid reaches the second consumable, a substantial amount of fluid may remain within the tube itself. This volume of fluid is known as a “dead volume” and may cause fluid to be wasted during a bioprocessing operation, particularly where large lengths of tubing are required to connect consumables. This is particularly problematic when only a small sample needs to be taken from a consumable, which may occur regularly during a bioprocessing operation.

Thus, for at least the reasons discussed above, until now it has been considered impractical to develop a bioprocessing system that utilises flexible tubes that are connected by automated tube welding. It is an object of the present invention to address the problems discussed above.

### SUMMARY OF INVENTION

According to a first aspect described herein there is provided a device for collecting a fluid sample from a fluid-containing consumable in a bioprocessing system, the consumable having a tube fluidly attached thereto, the device comprising: a fluid connection portion configured to be connected to the tube of a fluid-containing consumable, and to enable pumping of fluid or air through the device; and an air input portion configured to allow sterile air to be supplied to the device; a sampling portion connected to the fluid connection portion, wherein during pumping into the device, the sampling portion is configured to receive a fluid sample from the consumable via the fluid connection portion; a bypass tube having a first end connected to the fluid connection portion and a second end connected to the air input portion, wherein the bypass tube is configured to receive air supplied from the air input portion and direct the air out of the device via the fluid connection portion without passing through the sampling portion, whereby to purge fluid from the tube connected to the consumable, and means for restricting fluid flow through each of the sampling portion and bypass tube, such that fluid being pumped into the fluid connection portion passes only into the sampling portion and air pumped out of the fluid connection portion passes only through the bypass tube.

In this way, once fluid is pumped into the device from the fluid-containing consumable (e.g., using a peristaltic pump on the flexible tube connected to the consumable), a fluid sample may be collected in the sampling portion. Subsequently, in order to prevent excess fluid from being retained in the tube attached to the consumable, this excess fluid may be “purged” by reversing the pumping direction to return the fluid to the consumable. Since the bypass tube allows air to fill the tube to replace the fluid being purged, the fluid sample remains in the device while the excess fluid is removed. Therefore, the device allows a representative sample of fluid to be collected from a consumable without wasting a substantial amount of the fluid. Alternatively or additionally, the device may be used to perform fluid purging at other points during a bioprocessing operation (e.g. at points other than when samples are collected in the device). In at least one embodiment, the tube connected to the fluid-containing consumable may be a flexible (non-rigid) tube; alternatively, in another embodiment, the tube may be provided as, or form part of, a (rigid) fluidic manifold of the fluid-containing consumable. Accordingly, the terms “tube” and “tubing” may refer to any kind of fluid conduit that functions as a tube or tubing, whether rigid or flexible.

The device may further comprise a sampling tube having a first end connected to the fluid connection portion, and a second end connected to the air input portion, wherein the sampling portion is provided between the first end and the second end of the sampling tube.

In this way, the air that is displaced from the sampling portion when a sample is received may be removed from the device via the air input portion; this allows the sampling portion to be formed from substantially rigid material. Alternatively, the sampling portion may be a flexible bag which may expand as the fluid sample is received therein, thereby accommodating an increase in volume of the sampling portion without displacing air.

Preferably, the sampling portion is provided by a sample container disposed between the first end and the second end of the sampling tube. Alternatively, the sampling tube itself may have a length sufficient to retain the fluid sample.

The sample container may have a tapered shape. In this way, the fluid may enter the sampling container at the narrow end of the taper before filing towards the wider end of the taper. This allows the volume of the fluid sample to be determined with proportional accuracy for both small samples and larger samples of fluid.

The sampling portion may comprise an extraction port for removing the fluid sample from the sampling portion. The extraction port may be a needle free sampling port, or a Luer lock capped port.

The means for restricting fluid flow through the device may comprise a first check valve to allow fluid to flow (only) from the fluid connection portion into the sampling portion. The (first) check valve restricts the fluid sample from draining out of the sampling portion, such as under gravity. Furthermore, where the sampling portion is provided by the sampling tube connected to the air input portion, the first check valve prevents the air from purging the fluid sample from the sampling portion, and instead forces the air to flow from the air input portion to the fluid connection portion through the bypass tube.

Preferably, the check valve is located on the sampling tube between the sampling portion and the air input portion. In this way, the fluid sample may be collected in the sampling portion without passing through the (first) check valve, which allows the valve to be an air valve that operates when dry.

The means for restricting fluid flow through the device may comprise a second check valve to allow air to flow (only) from the air input portion to the fluid connection portion through the bypass tube.

The second check valve restricts air from flowing from the fluid connection portion towards the air input portion via the bypass tube.

Preferably, one or both of the check valves are configured to inhibit flow of fluid therethrough (e.g., to only allow air to pass therethrough). In this way, it is not possible for fluid to reach the air input portion through the device, and the device may be operated in any orientation within the bioprocessing system.

Alternatively, the means for restricting fluid flow through the device may comprise a pinchable portion of tubing on both the sampling portion and the bypass tube; in this way, either or both of the pinchable portions may be pinched closed in order to restrict fluid flow.

The fluid connection portion may comprise a length of (e.g. flexible) tube, thereby enabling connection of the device to the tube of the consumable by tube welding.

By using tube welding to connect the fluid connection portion to the consumable, a closed system is maintained throughout the sampling process, thereby reducing the likelihood of contamination of the bioprocessing system of the surroundings. Since tube welding may be used for other connections in the bioprocessing system, a separate connection process is not required for sampling.

The air input portion may be an air filter configured to supply sterilised air from the surroundings. Alternatively, the air input portion may comprise a container of sterile air. For example, the container may comprise a syringe or bag containing sterile air.

Preferably, the air input portion is a single air input portion connected to both the sampling portion and the bypass tube. Alternatively, the sampling portion and the bypass tube may be connected to separate air supplies, such as separate air filters or separate containers of sterile air. Preferably, where the air input portion is a single air input portion, the air input portion is connected to both the sampling tube and the bypass tube by a single tube (e.g., via a y-connector or splitter).

The device may further comprise an external housing. In this way, the device may be manipulated by a robotic device of the bioprocessing system. Furthermore, the device may be attached to other parts of the bioprocessing system (e.g., a bioprocessing apparatus) via the external housing; the external housing may have one or more engagement features to facilitate mounting of the device within the bioprocessing system and/or manipulation of the device by a robotic device. The external housing may hold parts of the device such as the fluid connection portion, the air input portion, and/or the extraction port at predetermined positions to facilitate consistent engagement by the robotic device.

According to another aspect described herein, there is provided a sampling cartridge comprising a housing configured to retain one or more of the devices as described above and herein. In this way, a robotic device may engage the cartridge to move it around the bioprocessing system, and when a sampling operation is performed, the robotic device may engage one of the individual devices retained in the housing.

According to another aspect described herein there is provided a method of collecting a fluid sample using the device as described above and herein, comprising: connecting the fluid connection portion to a tube connected to a consumable; pumping fluid from the consumable into the sampling portion of the device via the fluid connection portion; pumping fluid remaining in the tube back into the consumable, thereby drawing air from the air input portion into the tube via the bypass tube.

According to another aspect of the present invention there is provided a consumable for a bioprocessing system, the consumable comprising: a container for a fluid, a tube having a first end connected to the container and extending to a second (e.g., free) end, and an air supply fluidly connected to the tube at a location between its first and second end.

The air supply allows sterile air to be supplied to the tube. Advantageously, by providing a consumable with an air supply fluidly connected to the tube, air from the air supply may be used to purge fluid from the tube prior to performing any tube welds at (or near) the second (free) end of the tube. By removing fluid from the second end of the tube, the tube welds are performed on dry tubing, which is generally more reliable than “wet” welds (where fluid is still present), thereby improving reliability of connections to the consumable.

The air supply may comprise an air filter configured to supply sterilised air from the surroundings. The air supply may comprise a container of sterile air. The tube connected to the container may be a first tube, and the air supply may be connected to the first tube with a second tube, such as by welding. The (e.g., three-way) connection between the tube and the air supply may comprise one or more valves to control flow of fluid or air into the tube (such as from the container, the second end of the tube, and/or the air supply. For example, the one or more valves may be provided by a three-way valve or stopcock. Alternatively, the one or more valves may include two pinch valves (to allow for control of flow through either or both the tube and the air supply) or a single pinch valve and an (inline) check valve. The one or more valves may be integrated into the consumable and are preferably externally actuatable such as by a robotic device and/or an external solenoid.

Alternatively, control of flow (of fluid or air) through the (e.g., three-way) connection between the tube and the air supply may be performed externally. For example, during use, the consumable may be mounted in a bioprocessing apparatus with the first tube and/or the second tube retained in one or more valve seats. In this way during use, the valve seats may be actuated to restrict flow of fluid or air through either or both of the first tube connected to the container and/or the second tube connected to the air supply.

Optionally, the air supply may be provided using the device as described above and herein. The device may be connected to the tube by the fluid connection portion of the device. Advantageously, using the device as the air supply may allow both purging of fluid from the tube, and also sampling of fluid by the device.

According to another aspect described herein there is provided a fluid connection system for a bioprocessing apparatus, comprising: a consumable storage section having a first subsection and a second subsection, each subsection configured to store at least one fluid-containing consumable having a flexible tube fluidly connected thereto; a cell storage section configured to store a cell-containing consumable having at least a first flexible tube and a second flexible tube, each tube being fluidly connected to the cell-containing consumable; a tube connection section arranged between the consumable storage section and the cell storage section, the tube connection section comprising: a first portion configured to retain the first flexible tube of the cell-containing consumable and position it for connection with a flexible tube corresponding to a fluid-containing consumable stored in the first subsection of the consumable storage section; a second portion configured to retain the second flexible tube of the cell-containing consumable and position it for connection with a flexible tube corresponding to a fluid-containing consumable stored in the second subsection of the consumable storage section.

It will be appreciated that, while the cell-containing consumable is described above as having a first flexible tube and a second flexible tube, it may have more than two flexible tubes fluidly connected thereto, such as three or more flexible tubes. Similarly, the fluid-containing consumables may each have more than one flexible tube fluidly connected thereto, such as two or more flexible tubes. Since there may be multiple (e.g., four) fluid-containing consumables in the consumable storage section and multiple (e.g., two) tubes connected to the cell-containing consumable, the fluid connection system replaces the complex many-to-many tube connection configuration with two one-to-many tube connection configurations, which simplifies the connection process.

Furthermore, by having two subsections of the consumable storage section, where a respective portion of the tube connection section is arranged to connect one of the cell-containing consumable's flexible tubes to tubes in a corresponding subsection, it is possible to form connections between the fluid-containing consumables and the cell-containing consumable without crossing the tubes. This reduces the likelihood of tubes getting tangled with each other, and also keeps the tubes within a substantially 2-dimensional plane, thereby simplifying manipulation of the tubes by a robotic device. The cell-containing consumable may be referred to herein as a “cell consumable”. The cell-containing consumable may be cell culture chamber.

Preferably, the cell-containing consumable is a bioreactor, and the cell storage section is a bioreactor platform arranged to receive the bioreactor thereon. The bioreactor platform may comprise a shaker to agitate the bioreactor in order to resuspend the cells. Alternatively, the bioreactor platform may be a static platform.

Preferably, the tube connection section is further configured to arrange the first and second flexible tubes of the cell-containing consumable for connection with an air supply. In this way, the air supply may purge fluid from the first and second flexible tubes of the cell-containing consumable, thereby reducing waste of fluid/cells that remains in the tubes (e.g. from a sampling operation).

Preferably, the tube connection section is further configured to arrange the first and/or second flexible tube of the cell-containing consumable for connection with a sample collection device. The sample collection device may comprise at least one sample container into which fluid from the cell-containing consumable may be pumped to collect a representative sample.

Preferably, the tube connection section is configured to arrange the first and/or second flexible tube of the cell-containing consumable to a single device that provides both the air supply and the sample collection device. Preferably, the single device is the device as described above and herein.

The consumable storage section may comprise a plurality of consumable holding positions, preferably arranged in a linear configuration. The consumable holding positions are preferably evenly spaced. Preferably, each consumable holding position is provided by a slot, each slot configured to receive a fluid-containing consumable. Preferably, the fluid-containing consumable is housed inside a cartridge, and each slot comprises one or more engagement features to releasably retain the cartridge within the slot. Therefore, the fluid-containing consumable may be referred to herein as the “cartridge consumable”. Alternatively, the fluid-containing consumables may be hung upon a linear rack, where the consumable holding positions are defined by adjacent positions along the rack.

Preferably, the plurality of consumable holding positions includes an intermediate position that is included in both the first subsection and the second subsection of the consumable storage section. For example, where there are a total of five positions for fluid-containing consumables in the consumable storage section, the first subsection may comprise positions one to three, and the second subsection may comprise positions three to five. This arrangement still prevents crossing of tubes when connections from the intermediate position are made to either the first flexible tube of the cell-containing consumable or the second flexible tube of the cell-containing consumable. Alternatively, the subsections may be separate and non-overlapping, where positions for fluid-containing consumables cannot be in both the first and second subsections.

Preferably, the consumable storage section may comprise a third subsection configured to receive at least one fluid-containing consumable having a flexible tube fluidly connected thereto, wherein the third subsection includes a consumable holding position that is not part of either the first subsection or the second subsection. In other words, the consumable holding position of the third subsection (e.g., a “buffer” position) is not used for holding a consumable for connection to the cell-containing consumable. In this way, the buffer position may be used to swap a fluid-containing consumable held in the first subsection with a fluid-containing consumable held in the second subsection; this allows any of the fluid-containing consumables to be connected to either of the cell-containing consumable's flexible tubes, without any of the tube being crossed.

The tube connection section may comprise: a first plurality of tube clips, wherein each consumable holding position in the first subsection corresponds to at least one of the tube clips in the first plurality of tube clips; a second plurality of tube clips, wherein each flexible tube connected to the cell-containing consumable corresponds to at least one of the tube clips in the second plurality of tube clips.

In this way, the flexible tubes may be retained by the tube clips at predetermined positions in the tube connection section. Therefore, a robotic device may consistently engage the tubes based on the known positions of the tube clips in the tube connection section. Preferably, the tube clips retain the flexible tubes (within the first plurality and/or the second plurality) parallel and/or evenly spaced to each other.

Preferably, each position of the consumable storage section corresponds to a pair of tube clips (or more) in the first plurality of tube clips, and/or each flexible tube connected to the cell-containing consumable corresponds to a pair of tube clips in the second plurality of tube clips. In this way, when one of the tubes is retained by a pair of tube clips, the tube is retained between the tube clips along a predetermined axis. Since a length of the tube is retained along a predetermined (straight) axis, the tube may be more consistently engaged by the robotic device. For example, where the robotic device comprises a linear peristaltic pump, the pump may engage the tube along the predetermined axis of the tube between the pair of tube clips in order to pump fluid through the tube.

At least one of the tube clips may comprise: a tube seat having a profile shaped to receive a tube at a predetermined position on the tube seat; a retaining element movable relative to the tube seat between an open position for allowing the tube to be received by the tube seat, and a closed position for retaining the tube in the predetermined position on the tube seat; an actuator configured to move the retaining element between said open and closed positions; wherein the actuator is controllable to move the retaining element between a plurality of intermediate positions in each of which the retaining element applies a different force to the tube against the tube seat whereby to control fluid flow through the tube. The intermediate positions also allow different sizes of tube to be properly retained and pinched closed in the tube clip.

At least one of the tube clips may comprise at least one sensor to detect one or more of: the presence of a portion of tube in the tube clip, and/or whether a portion of tube retained within the tube clip contains fluid.

According to another aspect described herein, there is provided a bioprocessing apparatus comprising the fluid connection system as described above and herein. The consumable storage section, the cell storage section, and the tube connection section may be provided by the bioprocessing apparatus.

The bioprocessing apparatus may further comprise one or more of, a loading system, a storage system, an incubation system, and an unloading system.

According to another aspect described herein, there is provided a bioprocessing system comprising: the fluid connection system as described above and herein and/or the bioprocessing apparatus as described above and herein.

The bioprocessing system may further comprise a robotic device configured to manipulate a fluid connection between one or more flexible tubes of the bioprocessing system. The robotic device may comprise a robotic arm having a robotic end effector. The robotic device may comprise a tube welder for forming sterile connections between tubes, a peristaltic pump for pumping fluid through connected tubes, and/or a tube sealer for disconnecting and sealing tubes. The robotic device may be provided as a separate device from the bioprocessing apparatus, such as a mobile manipulation unit that may move relative to the bioprocessing apparatus. Alternatively, the robotic device may be provided as part of the bioprocessing apparatus.

The robotic device is configured to form connections between the tubes in the tube connection section. Preferably, the robotic device is configured to connect the first flexible tube of the cell containing consumable (e.g., only) with a flexible tube of a fluid-containing consumable in the first portion of the tube connection section, and to connect the second flexible tube of the cell containing consumable (e.g., only) with a flexible tube of a fluid-containing consumable in the second portion of the tube connection section. The robotic device only forms connections of tubes from the first subsection of the consumable storage section within the first portion of the tube connection section. The robotic device (e.g., only) forms connections of tubes from the second subsection of the consumable storage section within the second portion of the tube connection section.

In this way, the robotic device is operated so that tubes are not crossed when making connections, which reduces the likelihood of tangling of the tubes. The robotic device may be configured to perform a particular bioprocessing workflow, which defines steps (e.g., connection, pumping, disconnection and/or sealing steps) to be performed in a particular sequence by the robotic device.

According to another aspect described herein, there is provided a method of connecting a first flexible tube attached to a first consumable to a second flexible tube attached to a second consumable, the method comprising, using a robotic device: engaging the first flexible tube retained in a first tube clip, and pulling the first flexible tube through the first tube clip to arrange the first flexible tube adjacent to the second flexible tube retained in a second tube clip; while maintaining engagement of the first flexible tube with the robotic device, engaging the second flexible tube; welding the first flexible tube to the second flexible tube to form a (first) joined tube connecting the first consumable to the second consumable.

The robotic device may comprise a tube welder having a first pair of jaws and a second pair of jaws, such that the first flexible tube may be engaged by the first pair of jaws while the second flexible tube may be (simultaneously) engaged by the second pair of jaws (or vice versa). Once both tubes are engaged, the tube welder may pinch the tubes using the pair of jaws, heat and cut the tubes along a cutting plane (such as using a heated blade to melt the tubes during cutting), align the first flexible tube with the second flexible tube, and press the melted ends of the tubes together to form the joint tube. Preferably, the tube welder is provided on a robotic end effector of the robotic device.

The method may further comprise removing the second tube from the second tube clip before welding, and returning the joined tube into the second tube clip after welding. Advantageously, this provides more space for the robotic device to manipulate the tubes when welding the first flexible tube to the second flexible tube.

The method may further comprise: sealing the joined tube between the first tube clip and the second tube clip to divide it into a first flexible tube retained in the first tube clip and a second flexible tube retained in the second tube clip; engaging a third flexible tube retained in a third tube clip, the third flexible tube connected to a third consumable, and pulling the third flexible tube through the third tube clip to arrange the third flexible tube adjacent to either the first flexible tube or the second flexible tube; while maintaining engagement of the third flexible tube with the robotic device, engaging the first or the second flexible tube; welding the third flexible tube to the first or second flexible tube to form a second joined tube connecting the first or second consumable to the third consumable.

In this way, subsequent connections may be made between flexible tubes, where the tubes are always held by either a tube clip or the robotic device; therefore, the location and movement of all of the tubes is known at all stages when connections are made between consumables, thereby reducing the likelihood of incorrect connections being made.

According to another aspect described herein there is provided a tube clip for retaining a portion of flexible tube, comprising: a tube seat configured to receive a tube in a predetermined position; a retaining element movable relative to the tube seat between an open position for allowing the tube to be received by the tube seat in the predetermined position, and a closed position for retaining the tube in the tube seat; an actuator configured to move the retaining element between said open and closed positions; wherein the actuator is further configured to move the retaining element past the closed position whereby to pinch the tube, at least partially, closed against the tube seat, such that fluid flow through the tube can thereby be controlled. The tube clip may be provided in the fluid connection system as described above. The tube seat may be configured to receive the tube from a robotic device.

Accordingly, the actuator is controllable to move the retaining element between a plurality of intermediate positions in each of which the retaining element applies a different force to the tube against the tube seat whereby to control fluid flow through the tube and/or to allow different diameters of tube to be used with the tube clip. Advantageously, the tube clip may pinch the tube shut (e.g., occlude the tube) to prevent flow of fluid therethrough, may (tightly) retain the tube to restrict flow of fluid therethrough, may (lightly) retain the tube without substantially restricting flow of fluid therethrough, and/or may fully release the tube from the tube clip. The tube clip may lightly retain the tube with sufficient force to prevent longitudinal movement of the tube through the tube clip; the tube clip may also lightly retain the tube to allow longitudinal movement through the tube clip.

The following preferable features may be provided with either or both of the fluid connection system as described above and herein, or the tube clip as described above and herein.

Preferably, the actuator is controllable to provide a continuous range of intermediate positions of the retaining element. Optionally, the actuator may be controllable to apply a continuous range of forces to the tube against the tube seat.

The profile of the tube seat may be a semi-circular or substantially C-shaped, may be rectangular or may be a generally V-shaped notch or groove. In this way, when force is applied to the tube against the tube seat, the tube is urged towards the predetermined position, thereby facilitating consistent engagement of the tube in the tube clip by an external robotic device.

The retaining element may comprise a retaining arm arranged to enclose the tube within the tube seat when the retaining element is in the closed position. The retaining arm may be a hooked element, a bar element, or any other suitable shaped arm for retaining the tube against the tube seat. One or more additional retaining arms may be used, such as two bar elements on two separate pivots.

The tube seat may comprise a first portion and a second portion that are spaced apart in a direction parallel to an axis of the tube with a gap within which the retaining arm is arranged to contact the tube. In this way, the retaining arm applies a symmetrical force to the tube so that it is not bent during engagement.

Preferably, the actuator is configured to rotate the retaining element during movement from the open position and the closed position. In other words, the retaining element may be attached to a pivot about which the retaining element rotates. In this way, when the retaining element is in the open position, the tube seat may be fully exposed, thereby allowing the tube to be unobstructed when received in the tube seat.

The tube clip may further comprise a camming element rotatably coupled to the actuator, whereby rotation of the camming element both rotates the retaining element and moves the retaining element laterally during movement from the open position to the closed position. In this way, during initial movement of the retaining element from the open position to the closed position, the retaining element may rotate to bring the retaining arm in front of the tube seat. Subsequently, during subsequent movement of the retaining element, the camming element causes the retaining arm to move laterally towards the tube in a substantially linear direction; this reduces shear forces applied to the tube, (particularly if the tube is pinched shut when in the closed position), which may reduce the risk of the tube breaking.

Preferably, the retaining element is biased by a spring to oppose the force applied by the actuator, thereby returning the retaining element to the open position when the actuator is disengaged. In this way, the actuator does not need to be controlled to actively rotate the retaining element towards the open position.

Preferably, the tube clip is configured to enable longitudinal movement of the tube therethrough. As used herein, longitudinal movement of the tube preferably refers to movement of the tube along the axis of the tube. By enabling longitudinal movement of the tubes, the position of at least part of the tube remains known even when other parts of the tube are being moved (e.g., to connect to other tubes). In this way, every tube may always be located using at least one tube clip in the tube connection section. The tube clip may comprise a roller to enable longitudinal movement of a retained tube. Preferably, the retaining element is movable to a position in which the tube is retained within the tube clip but not gripped or pinched by the retaining element. In this way, the tube does not necessarily have to contact the tube clip when in the closed position, thereby enabling the tube to be easily pulled longitudinally through the tube clip while still keeping the tube at a predetermined position in the bioprocessing system (thereby enabling engagement by a robotic device). A tube contacting surface of the retaining element and/or the tube seat may comprise a low-friction surface so as not to substantially inhibit longitudinal motion of the tube through the tube clip.

The tube clip may further comprise at least one sensor to detect one or more of: the presence of a portion of tube in the tube clip, and/or whether a portion of tube retained within the tube clip contains fluid.

The at least one sensor may include a bubble sensor. The sensor may comprise a capacitive and/or an ultrasonic sensor. The sensor may comprise a force sensor to measure the force or torque applied to the tube by the retaining arm. Preferably, a plurality of sensors are provided.

The tube clip may further comprise a tube guide arranged to support the tube at a predetermined position along the (longitudinal) axis of the tube that is offset from the tube seat.

In this way, the position of the tube is not only known precisely at the tube seat, but is also known along a length extending between the tube seat and the tube guide. This may enable more consistent engagement of the tube by an external robotic device. The tube guide may comprise a tube fin arranged to extend along the (longitudinal) axis of the tube. This allows for easy engagement of the tube by the robotic device, since the fin supports the tube and is thin enough not to block the jaws of the robotic device from closing when the jaws engage the tube. The tube guide may comprise a second tube seat that is offset from the (e.g., first) tube seat along the longitudinal axis. In this way, the position of the tube is known at both the first and second tube seats as well as the axis that extends therebetween, thereby allowing the tube to be engaged by a robotic device without being obstructed by either of the tube seats.

It will be understood by a skilled person that any apparatus feature described herein may be provided as a method feature, and vice versa. It will also be understood that particular combinations of the various features described and defined in any aspects described herein can be implemented and/or supplied and/or used independently.

Moreover, it will be understood that the present invention is described herein purely by way of example, and modifications of detail can be made within the scope of the invention. Furthermore, as used herein, and “means plus function” features may be expressed alternatively in terms of their corresponding structure.

### BRIEF DESCRIPTION OF DRAWINGS

One or more embodiments will now be described, purely by way of example, with reference to the accompanying figures, in which:

FIG. 1 shows an example of a bioprocessing apparatus that may form part of a bioprocessing system;

FIG. 2A shows cartridge containing a cartridge consumable having a tube connected thereto, and FIG. 2B shows a cell culture chamber having a plurality of tubes connected thereto;

FIG. 3 shows a first example of a fluid connection system for connecting a plurality of cartridge consumables to the cell culture chamber;

FIG. 4A shows a preferred example of a fluid connection system;

FIG. 4B shows a tube connection section of the fluid connection system of FIG. 4A;

FIGS. 5A to 5D show steps in a method of connecting tubes using the fluid connection second of FIG. 4B;

FIGS. 6A to 6D show alternative fluid connection systems;

FIG. 7A to 7C show examples of a sampling device that also enables fluid to be purged from tubes in the bioprocessing system;

FIGS. 8A and 8B show an example of a tube clip that may be used in the fluid connection system and tube connection section of FIGS. 4A and 4B; and

FIGS. 9A to 9G show alternative ways that samples may be extracted.

### DETAILED DESCRIPTION

### Overview

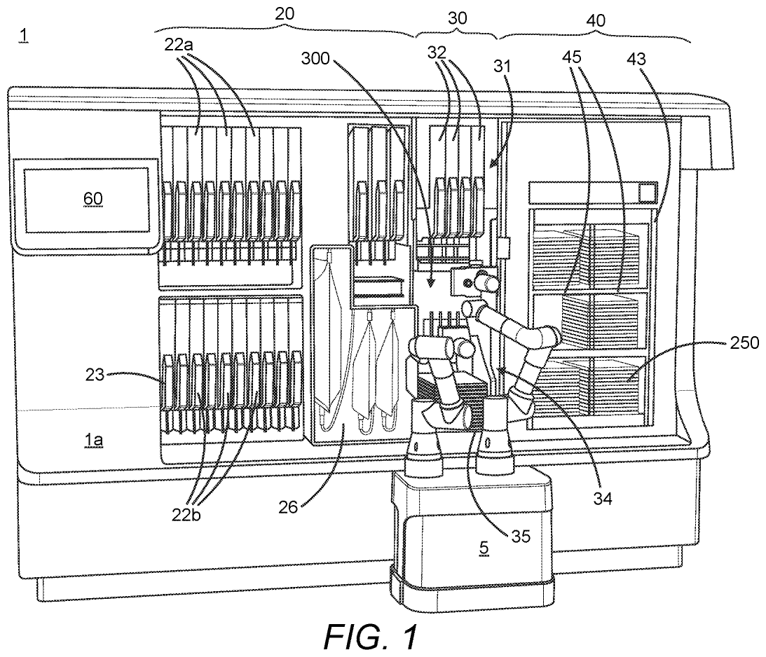

FIG. 1 shows a bioprocessing apparatus 1. The bioprocessing apparatus 1 may be part of a wider bioprocessing system, such as a processing station of a bioprocessing system. The bioprocessing apparatus 1 may have a plurality of sub-systems, such as a loading system, a storage system 20, a fluid connection system 30, an incubator system 40, and an unloading system. In the example shown in FIG. 1, the loading system and the unloading system have been omitted. While FIG. 1 shows each system at a particular location on the apparatus 1, it will be appreciated that the locations are purely exemplary and that the system can be located in other positions.

The bioprocessing apparatus 1 is operable to receive a plurality of consumables and manipulate the consumables in order to perform a bioprocessing operation. The bioprocessing operation may include steps such as transfer of fluid between the consumables, incubation of consumables, and sampling from consumables, for example.

The consumables each have at least one flexible tube fluidly connected thereto; as will be described later in more detail, transfers of fluid between consumables occurs by connecting consumables together via their respective tubes before fluid is pumped therebetween. More specifically, the corresponding tubes are welded together using a tube welder, fluid is pumped using a peristaltic pump, and the tubes are disconnected again with a tube sealer. When tubes are not connected together, the free end of each tube remains sealed so that the contents of each tube and the consumable are never exposed to the surroundings; this provides a “closed system” and prevents contamination of the contents of the containers or the surroundings.

The inputs to the bioprocessing apparatus 1 may be consumables containing reagents, media, cytokines and patient samples such as cells, and empty consumables such as for collecting samples for testing. Following the bioprocessing operation, the outputs of the bioprocessing apparatus 1 may be consumables containing a cell culture, waste consumables, or empty consumables, for example.

The particular steps of the bioprocessing operation may be defined by a “bioprocessing workflow”. All of the steps of the bioprocessing operation are preferably automated, whereby the manipulation of the consumables (including fluid connections and transfers) is performed by a robotic device 5, preferably without any operator intervention. The robotic device 5 may have at least one robotic arm with at least one end effector having components such as the tube welder for forming sterile connections between tubes, the peristaltic pump for pumping fluid through connected tubes, and/or the tube sealer for disconnecting and sealing tubes. These components may be provided on the same end effector, or may be provided on separate end effectors or separate robotic devices.

The tube welder may have a first pair of jaws for gripping a first tube and a second pair of jaws for gripping a second tube. The peristaltic pump is preferably a linear peristaltic pump with a plurality of individual pressing elements that compress a retained tube to effect a peristaltic pumping action to pump fluid through the tube. The tube sealer may comprise an RF sealer or a heat sealer; alternatively, the sealing may be performed by the tube welder.

As shown in FIG. 1, each of the sections is accessible from a front surface 1a of the bioprocessing apparatus 1. This allows the robotic device 5 to manipulate the consumables; for example, as shown in FIG. 1, the robotic device 5 may be separate to the apparatus 1, such as a mobile manipulation unit that may move freely upon a factory floor where the bioprocessing apparatus 1 is located. Alternatively, the robotic device 5 may be part of the bioprocessing apparatus 1, such as mounted upon a rail that runs adjacent to the front surface 1a.

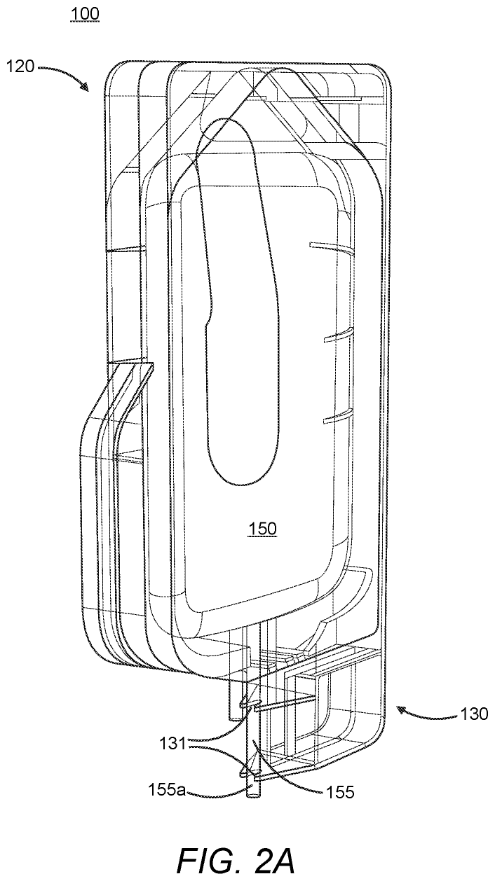

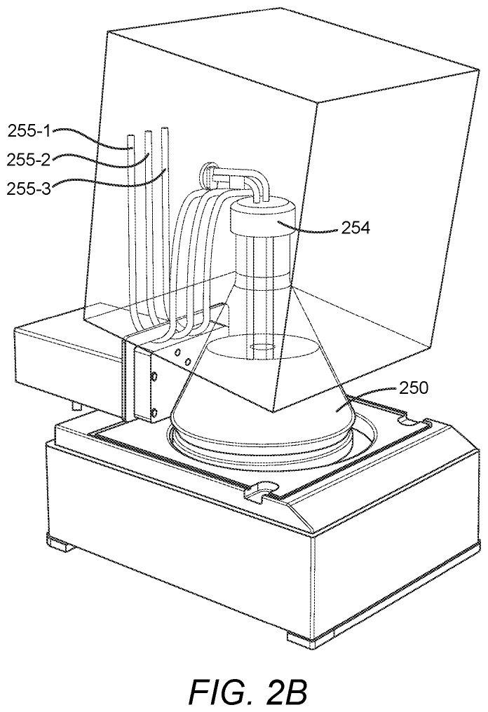

The term “consumable” is used to refer collectively to components that directly contain and/or contact biological material, and therefore cannot be reused. More specifically, consumables may be containers such as bags or jars. To facilitate handling and manipulation of the consumables, the consumables may be located within cartridges 100, which will now be described in relation to FIGS. 2A and 2B.

FIG. 2A shows an example of a cartridge 100 to hold a consumable 150. The consumable 150 held in the cartridge 100 may be referred to as a “cartridge consumable” 150. The cartridge 100 comprises a first portion 120 for holding the cartridge consumable 150, and a second portion 130 for retaining the flexible tube 155 at least in part along a predetermined path. More specifically, the first portion 120 is provided by a housing 120 within which the cartridge consumable 150 (in the form of a bag 150) is located. The second portion 130 may comprise one or more tube clips 131 to retain a free end 155a of the tube 155 in a fixed position relative to the cartridge 100 (and therefore relative to the slots), which enables the tube 155 to be engaged at consistent positions by the robotic device 5. For example, the tube 155 may engaged at a position directly between two tube clips 131. Alternatively, the cartridge 100 may only have a single passive clip 131. Preferably, the cartridge 100 is configured to dispense and retract the tube 155 connected to the consumable 150; in this way, the cartridge 100 can accommodate the changing length of the tubes 155, 255 due to repeated connections being made.

FIG. 2B shows an example of a cell consumable 250 (e.g., a cell culture chamber 250). The cell consumable 250 may be referred to as a “bioreactor” 250. The cell consumable may be a gas permeable cell culture vessel. In this example, the cell consumable 250 is generally shaped as a conical flask with an opening at its top, though this shape is only exemplary. The cell consumable 250 may be any pot, flask or container that contains cells. A removable cap 254 is (e.g. screw) mounted to the opening of the cell consumable 250. A plurality of tubes 255 extend through the cap 254 to allow fluid to be pumped into and/or out of the cell consumable 250. For example, a first tube 255-1 may extend to the top of the cell consumable 250, and a second tube 255-2 may remain towards the base of the cell consumable 250; this allows fluid to be easily added at the top of the cell consumable 250 and removed from the base of the cell consumable 250. Optionally further tubes may be present, such as a third tube 255-3. It will be appreciated that the tubes 255 may be configured differently and that more or fewer tubes 255 may be present.

The tubes 255 extend from the cell consumable in order to allow the tubes 255 to be accessed by the robotic device 5. In this way, fluid may be added and/or removed from the cell consumable 250 via the tubes 255. The free ends of the tubes 255a are arranged to have a fixed position relative to the cell consumable 250, such as by using one or more passive clips (either on the cell consumable 250 or on the fluid connection system 30); in this way, when the bioreactor 250 is mounted within the bioprocessing system 1, the tubes 255 may be engaged at consistent positions by the robotic device 5. While the bioreactor 250 is shown in FIG. 2B as being located inside an external incubator with a base and a cover, it will be appreciated that these components are not required.

As will be described later in relation to the fluid connection system 30, during a bioprocessing operation, flexible tubes 155 corresponding to the cartridge consumables 150 (referred to as “cartridge consumable tubes 155”) may be welded to the flexible tubes 255 corresponding to the cell consumable 250 (referred to as “cell consumable tubes 255” or “bioreactor tubes”).

While not shown in FIG. 1, the (optional) loading system will now be described. The loading system includes a plurality of loading slots. Each loading slot is configured to receive a cartridge 100 holding a cartridge consumable 150, such as a cartridge consumable 150 containing a patient sample or reagent. The housing 120 of the cartridge 100 may have a standardised shape and/or one or more engagement components to facilitate consistent insertion of the cartridge 100 into one of the loading slots. The cartridges 100 may be manually loaded by a human operator or loaded by an automated robotic device 5. The loading system also includes at least one bioreactor loading platform, where a cell consumable 250 may be initially loaded into the apparatus 1. As described previously, the cell consumable 250 has a plurality of flexible tubes 255 facilitate transfer of fluid to and from the cell consumable 250.

The apparatus 1 comprises a storage system 20. The storage system 20 includes a plurality of storage slots 22. Once loaded into the loading slots, the robotic device 5 may transfer the cartridges 100 into the storage slots 22. Alternatively, the cartridges 100 may be loaded directly into the storage slots 22 (i.e., no loading system is required). In this example, the storage slots 22 comprise a first plurality of storage slots 22a for room temperature storage, and a second plurality of storage slots 22b within a refrigerator 23. In this way, many different types of cartridge consumables 150 may be stored within the bioprocessing apparatus 1 within standardised cartridges 100, such as cartridge consumables 150 containing different reagents, media or patient samples such as cells; when required, any cartridge 100 may be removed from one of the storage slots 22 and moved to the fluid connection system 30 for fluid to be transferred. The storage system 20 may have other sections, such as a media storage section 26 for large media bags; this allows media to be loaded into the apparatus 1 in bulk and subsequently transferred into cartridges 100 (rather than smaller bags of media needing to be constantly provided in cartridges 100).

The fluid connection system 30 will now be described. During a bioprocessing operation, fluid may need to be transferred between consumables, such as from one or more of the cartridge consumables 150 into the cell consumable 250, or vice versa. The fluid connection system 30 comprises a consumable storage section 31 comprising a plurality of transfer slots 32; for a transfer operation, cartridges 100 may be moved by the robotic device 5 from the storage slots 22 in the storage system 20 to the transfer slots 32 of the fluid connection system 30. The fluid connection system 30 also comprises a cell consumable storage section 34. The cell consumable storage section 34 may comprise a bioreactor platform 35. For a transfer operation, a bioreactor 250 may be moved onto the bioreactor platform 35, such as from the bioreactor loading platform of the loading system.

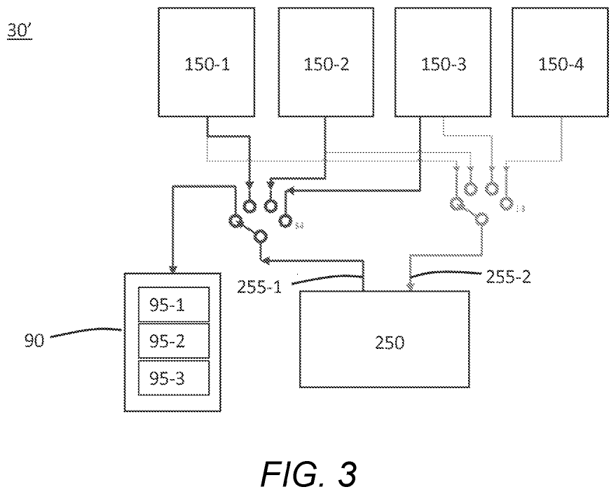

Once the cartridges 100 are correctly loaded into the fluid connection system 30, the tube 155 corresponding to each cartridge consumable 150 is located at a predetermined position relative to each transfer slot 32. Similarly, when the cell consumable 250 is correctly loaded into the fluid connection system 30, the tubes 255 connected to the cell consumable 250 are located at predetermined positions relative to the bioreactor platform 35. In this way, the robotic device 5 may reliably engage any of the tubes 155, 255 connected to any of the consumables 150, 250 in order to form connections therebetween. Further features of the fluid connection system 30 will be discussed later in relation to FIGS. 3 to 6.

The incubator system 40 will now be described in more detail. Once various reagents have been mixed together within the cell consumable 250 (such as following a fluid transfer occurring in the fluid connection system 30), the cell consumable 250 may be moved into the incubator system 40 for cell expansion. Since the bioprocessing apparatus 1 may perform several bioprocessing operations (i.e. for different patient samples) in parallel, the incubator system 40 preferably comprises several incubation platforms 45 each arranged to support a respective cell consumable 250. The incubator system 40 enclose the cell consumables 250 within an incubation chamber 43 to maintain the cell consumables 250 at a temperature higher than room temperature, such as about 37 degrees. The incubator system 40 may also be configured to agitate the contents of the cell consumables 250, such as to shake or tilt the cell consumables 250. In particular, each incubation platform 45 may be individually operable to agitate a cell consumable 250 placed thereon.

The cell consumables 250 may be transferred back to the fluid connection system 30 from the incubation system 40 at several points during a bioprocessing workflow. For example, samples may be extracted from the cell consumable 250 in order to monitor the cell growth.

While not shown in FIG. 1, the (optional) unloading system will now be described in more detail. The unloading system includes a plurality of unloading slots, which are similar to the loading slots, storage slots 22, and transfer slots 32 previously described. The unloading slots receive cartridges 100 that have already been used, such as cartridges 100 with cartridge consumables 150 containing waste media, samples for testing, enriched cell culture, or empty cartridges. In this way, the used cartridges 100 may be easily removed from apparatus 1 via the unloading system. Alternatively, the cartridges 100 may be removed directly from the storage system 20 or the fluid connection system 30.

The unloading system also includes at least one bioreactor unloading platform, which may be similar to the bioreactor loading platform of the loading system. In this way, used cell consumables 250 may be easily removed from the apparatus 1 via the unloading system 50. Alternatively, the cell consumables 250 may be removed directly from the storage system 20 or the fluid connection system 30.

The bioprocessing apparatus 1 comprises a user interface 60. In this example, the user interface 60 is a tablet connected to the bioprocessing apparatus 1, though it will be appreciated that the user interface 60 may be provided elsewhere, including remotely to the apparatus 1. The user interface 60 may be used by a human operator when loading the cartridges 100 into the loading system; for example, the operator may identify the contents of the cartridges 100 loaded into the loading slots. By storing this information in the apparatus 1, it is possible to track the location of all of the cartridges 100 when moved around the apparatus 1 (e.g., between the slots, 22, 32). The user interface 60 may allow a user to input a particular bioprocessing workflow to be followed, or to instruct the apparatus 1 to perform a sampling operation.

### Fluid Connection System

The fluid connection system 30 will now be described in more detail. As outlined above, the fluid connection system 30 enables connections to be formed between the tubes 155 corresponding to the cartridge consumables 150 and the tubes 255 corresponding to the cell consumable 250. Typically, four cartridges 100-1, 100-2, 100-3, 100-4 will be present in the transfer slots 32, each holding a corresponding cartridge consumable 150-1, 150-2, 150-3, 150-4 connected to a corresponding tube 155-1, 155-2, 155-3, 155-4. The cell consumable 250 will have at least two tubes 255-1, 255-2.

Ideally, a fluid connection system should be able to perform the following:

Firstly, the fluid connection system should be able to connect any of the tubes 155 corresponding to any of the cartridge consumables 150 to either of the tubes 255 corresponding to the cell consumable 250.

Secondly, the fluid connection system should measure the volumes of fluid transferred between each of the consumables 150, 250 throughout the bioprocessing workflow.

Thirdly, since the tubes 155 typically extend downward from the cartridges 100, the fluid connection system should be able to prevent fluid from draining from the cartridge consumables 150 under gravity.

Fourthly, the fluid connection system should enable samples to be transferred from the cell consumable 250 to sample containers. This allows progress of the cell expansion in the cell consumable 250 to be monitored by taking representative samples throughout the bioprocessing method.

Fifthly, due to the lengths of tubing that extend between the consumables 150, 250, the fluid connection system should be able to purge any fluid remaining in the tubes 155, 255 after a transfer operation. For example, when fluid is pumped from a cartridge consumable 150 to the cell consumable 250 through a connected tube, even once sufficient fluid is transferred to the cell consumable 250, additional fluid will initially remain within the connected tube. If this fluid is not removed and instead left within the tube for an extended period, then the fluid may expire and contaminate other fluids within the closed system. Furthermore, the purged fluid is preferably returned to its original consumable, rather than being pumped into a waste container. Where samples are taken in sample containers, the tubes also need to be purged in a similar way.

In practice, a fluid connection system that can perform all of the above functions is difficult to achieve.

FIG. 3 shows a schematic diagram of a fluid connection system 30′. As shown, the cell consumable 250 is connected to a first tube 255-1 and a second tube 255-2. Furthermore, a sampling unit 90 is provided comprising a plurality of sample containers 95. In order to allow both of the tubes 255-1, 255-2 to be connected to the cartridge consumables 150 and the sampling unit 90, the fluid connection system 30′ requires the tubes 155, 255 to cross over each other at several points.

Indeed, the fluid connection system 30′ of FIG. 3 requires a many-to-many connection configuration, which requires the tubes to cross in at least one location.

However, crossing of tubes is problematic where the bioprocessing workflow is automated. Firstly, where several connections and disconnections are made, the same pair of tubes may be crossed repeatedly leading to tangling of the tubes. Secondly, crossing of tubes requires to the tubes to move out of a substantially 2-dimensional plane, which makes reliable engagement of the tubes (at consistent locations) difficult for a robotic device. Thirdly, only the first cell consumable tube 255-1 is able to connect to the sampling unit 90, so it is not possible to sample from the second cell consumable tube 255-2.

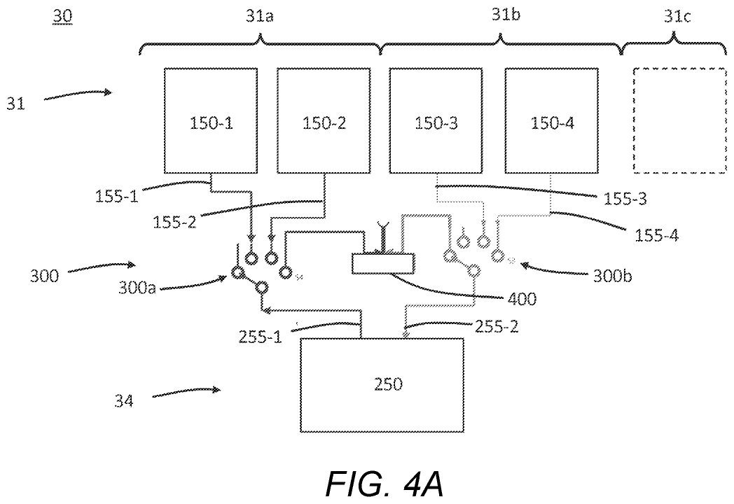

In order to address the problems discussed above, FIG. 4A shows a preferred embodiment of a fluid connection system 30. The system 30 comprises a consumable storage section 31 which has a first subsection 31a and a second subsection 31b. Each subsection 31a, 31b is configured to store at least one fluid-containing consumable 150 having a flexible tube 155 fluidly connected thereto. More specifically, as already described above the consumable storage section 31 has a plurality of slots 32, where each slot 32 is configured to receive a cartridge 100 having a cartridge consumable 150 held therein. The slots 32 are preferably arranged in a linear configuration with the slots 32 being evenly spaced. The slots 32 are divided into a first plurality of slots 32a and a second plurality of slots 32b, each corresponding to a respective subsection 31a, 31b of the storage section 31.

The fluid connection system 30 also includes a cell storage section 34 configured to store a cell consumable 250 (e.g., a cell culture chamber 250) having at least a first flexible tube 255a, 255b fluidly connected thereto.

The fluid connection system 30 also includes a tube connection section 300 arranged between the consumable storage section 31 and the cell storage section 34. Rather than allowing for connections between any of the cartridge consumables 150 to either of the tubes 255-1, 255-3 connected to the cell consumable 250, the tube connection section 300 comprises a first portion 300a and a second portion 300b. The first portion 300a is configured to retain the first flexible tube 255-1 of the cell consumable 250 and position it for connection with a flexible tube 155-1, 155-2 corresponding to a cartridge consumable 150 stored in the first subsection 31a of the consumable storage section 31. The second portion 300b is configured to retain the second flexible tube 255-2 of the cell consumable 250 and position it for connection with a flexible tube 155-3, 155-4 corresponding to a cartridge consumable 150 stored in the second subsection 31b of the consumable storage section 31.

Advantageously, by having a first portion 300a and a second portion 300b of the tube connection section 300, with each arranged to connect tubes in one of the subsections 31a, 31b of the consumable storage section, to one of the cell consumable's tubes 255-1, 255-2, it is possible to form connections between the consumables 150, 250 without crossing the tubes 155, 255. This reduces the likelihood of tubes 155, 255 getting tangled with each other, and also keeps the tubes 155, 255 within a substantially 2-dimensional plane, thereby simplifying manipulation of the tubes 155, 255 by the robotic device 5. While not shown in FIG. 4A, the consumable storage section 31 may have a slot 32 at an intermediate position which is in both the first subsection 31a and the second subsection 31b. The consumable storage section 31 may have a third subsection 31c, which is preferably provided by a single slot 32. In this way, where a tube 155-1, 155-2 from one of the cartridge consumables 150-1, 150-2 in the first subsection 31a needs to be connected to the second cell consumable tube 255-2 (or where a tube 155-3, 155-4 from one of the cartridge consumables 150-3, 150-4 in the second subsection 31b needs to be connected to the first cell consumable tube 255-1), the slot 32 in the third subsection 31c may be used to exchange two of the cartridge consumables 150 between the first subsection 31a and the second subsection 31b. In other words, the third subsection 31c provides a buffer position for one of the cartridge consumables 150 so that only one cartridge consumable 150 needs to be engaged at a time in order to perform a swap. By allowing swapping of cartridge consumables 150 between the first subsection 31a and the second subsection 31b, any cartridge consumable 150 can be connected to either of the cell consumable tubes 255-1, 255-2 without crossing the tubes.

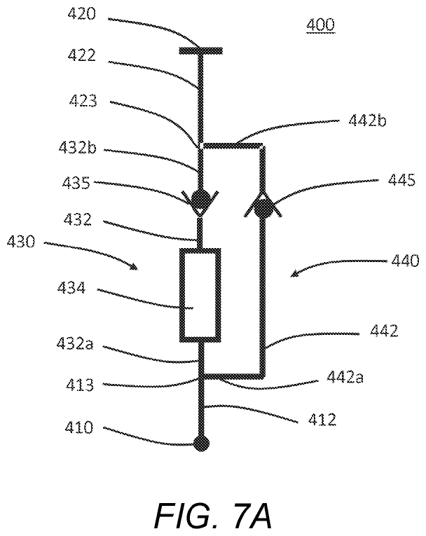

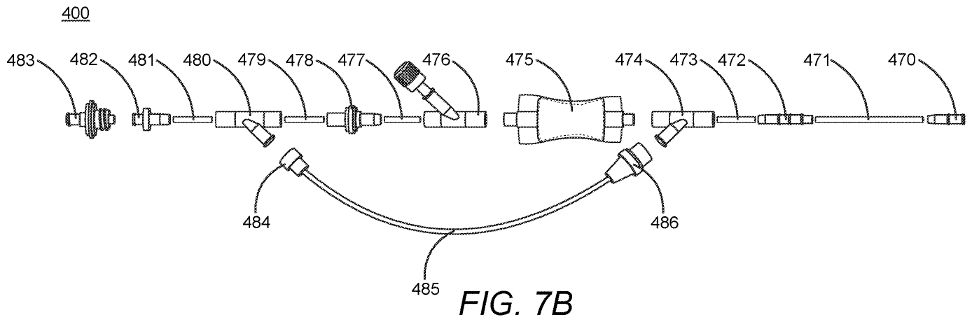

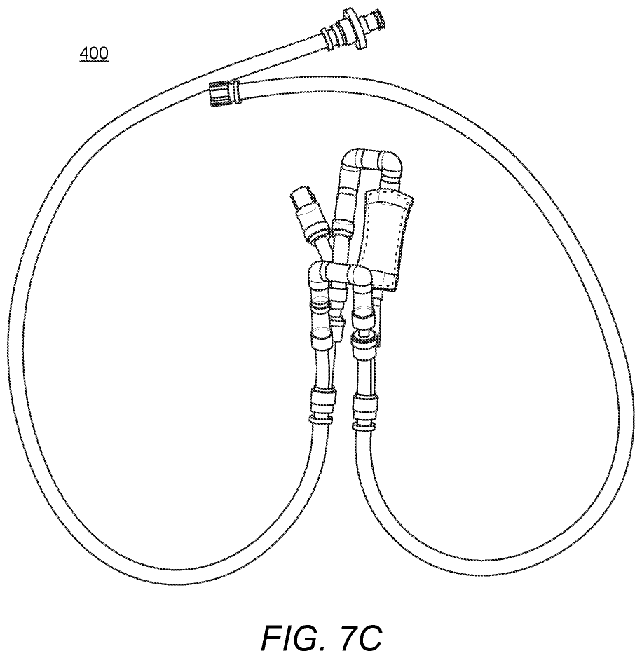

The fluid connection system 30 also includes a sampling device 400, which allows samples to be taken from the cell consumable 250. A particular example of a sampling device 400 will be described later in relation to FIGS. 7A-7C. Briefly, the sampling device 400 allows both for samples to be taken from the cell consumable 250 and also includes an air input portion 420 that allows any fluid remaining in the tubes 255-1, 255-2 after a sampling operation to be returned to the cell consumable 250.

FIG. 4B shows the tube connection section 300 in more detail. In this example, the tube connection section 300 is a tube connection panel 300 which is located in the fluid connection system 30 between the consumable storage section 31 (holding the cartridge consumables 150) and the cell storage section 34 (holding the cell consumable 250). During use, a plurality of tubes 155-1, 155-2, 155-3, 155-4, 155-5 may extend over a top edge 300a of the tube connection panel 300, each tube 155 corresponding to a cartridge consumable 150 in one of the slots 32 of the consumable storage section 31. Likewise, a plurality of tubes 255-1, 255-2, 255-3 may extend over the bottom edge 300b of the tube connection panel 300, each tube 255 connected to the cell consumable 250 in the cell storage section 34.

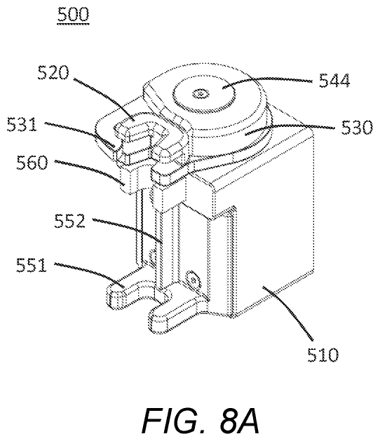

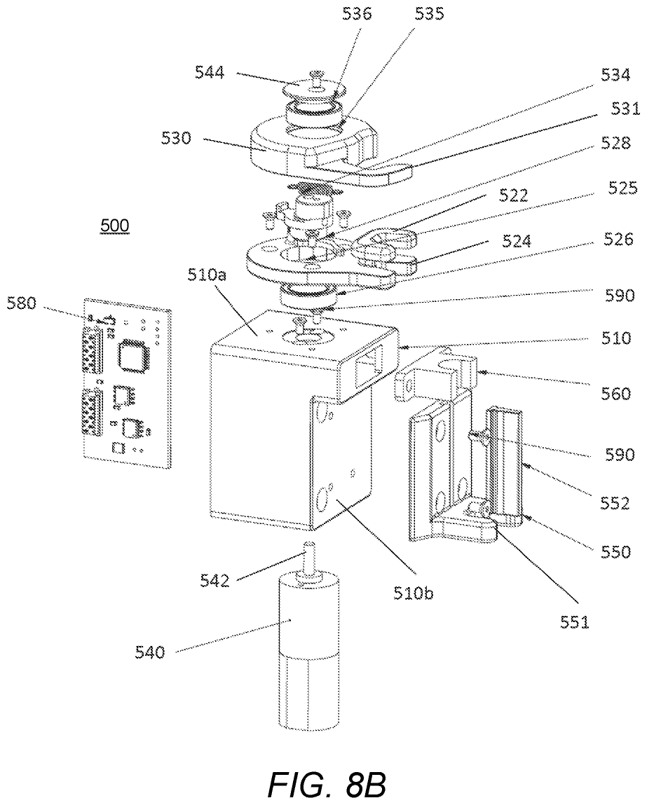

The tube connection section 300 comprises a plurality of tube clips 500 (only some labelled). A particular example of a tube clip 500 will be described in relation to FIGS. 8A and 8B. The tube clips 500 include a first plurality of tube clips 500a, the first plurality 500a being located adjacent to the top edge 300a of the tube connection panel 300; the first plurality of tube clips may be referred to as the “cartridge consumable tube clips 500a”. In this way, each tube 155 corresponding to one of the cartridge consumables 150 may be retained by at least one of the cartridge consumable tube clips 500a at predetermined positions in the tube connection section 300. More specifically, the first cartridge consumable tube 155-1 is retained by at least a first cartridge consumable tube clip 500a-1. Preferably, the first cartridge consumable tube 155-1 is retained by a first pair of cartridge consumable tube clips 500a-1, 500a-1′ (e.g., an “upper” cartridge consumable tube clip 500a-1 and a “lower” cartridge consumable tube clip 500a-1′). Similarly, each of the second to fifth cartridge consumable tubes 155 are retained by at least one cartridge consumable tube clip and preferably a pair of cartridge consumable tube clips, though for clarity not all the tube clips have been labelled. By using a pair of tube clips 500a-1, 500a-1′ the tube 155-1 is retained between the pair of tube clips along a predetermined axis. This allows the tube 155-1 to be more consistently engaged by the robotic device 5, since the location of the tube 155-1 is not only known at a single point, but along a continuous length of the tube 155-1.

The tube clips 500 include a second plurality 500b, the second plurality 500b being located adjacent to the bottom edge 300b of the tube connection panel 300; the second plurality of tube clips may be referred to as the “cell consumable tube clips 500b”. In this way, each tube 255 connected to the cell consumable 250 may be retained by at least one of the cell consumable tube clips 500b at predetermined positions in the tube connection section 300. More specifically, the first cell consumable tube 255-1 is retained by at least a first cell consumable tube clip 500b-1. Preferably, the first cartridge consumable tube 255-1 is retained by a first pair of tube clips 500b-1, 500b-1′ (e.g. an “upper” cell consumable tube clip 500b-1 and a “lower” cell consumable tube clip 500b-1′). Similarly, each of the second (and third) cell consumable tubes 255 are retained by at least one cell consumable tube clip and preferably a pair of cell consumable tube clips, though for clarity not all the tube clips have been labelled. The distance between pairs of tube clips in the second plurality 500b may be greater than the distance between pairs of cell consumable tube clips; this may allow additional space for a linear peristaltic pump to be arranged along the axis of the tube between the pairs of cell consumable tube clips.

While not shown in FIG. 4B, the sampling device 400 may be mounted on the tube connection section 300 at a central position that enables any of the cell consumable tubes 255-1, 255-2 to be connected thereto. For example, the sampling device may be provided in an external frame that is held by one of the tube clips 500. As described later, the sampling device 400 enables both sampling and purging through either of the cell consumable tubes 255-1, 255-2.

A method of connecting two tubes using the tube connection section 300 will now be described. In this example, the first cell consumable tube 255-1 is connected to the first cartridge consumable tube 155-1, though it will be appreciated that the method may be used to connect other tubes 155, 255.

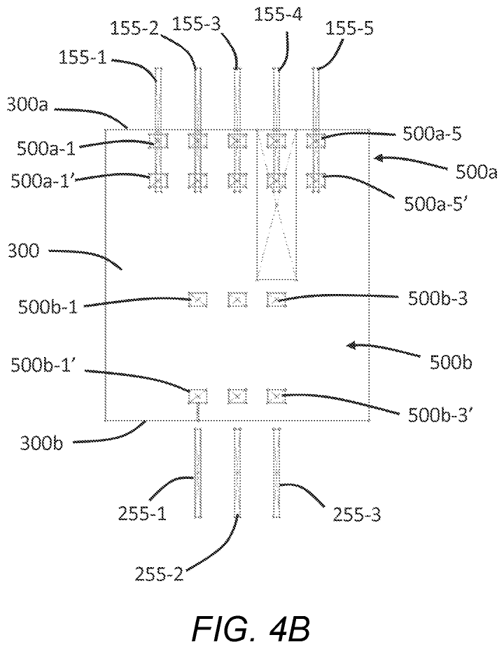

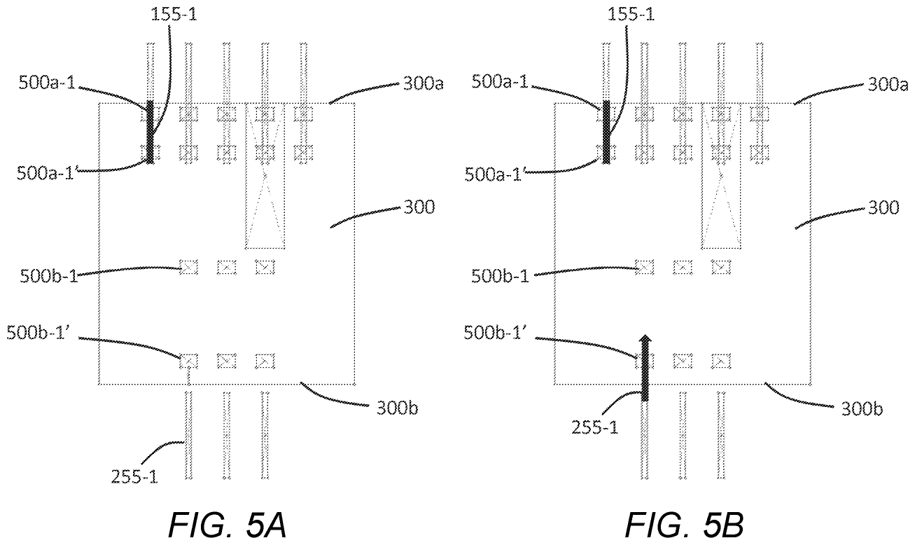

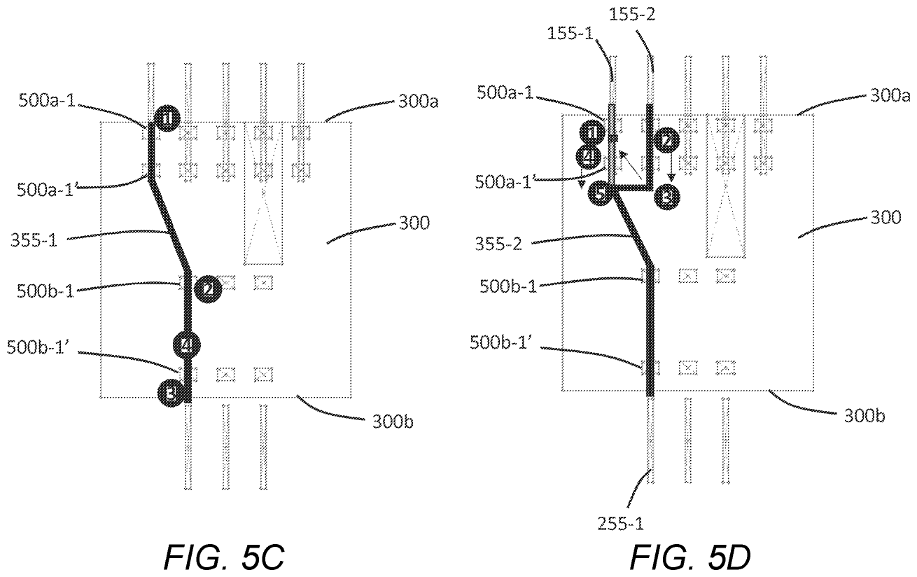

In FIG. 5A, the robotic device 5 engages the first cartridge consumable tube 155-1 with one of the pairs of jaws of the end effector, unclips it from the passive clip in its respective cartridge 100-1, pulls the tube 155-1 over the top edge 300a of the tube connection section 300 and clips it into the first pair of cartridge consumable tube clips 500a-1, 500a-1′.

In FIG. 5B, the robotic device 5 engages the first cell consumable tube 255-1 with one of the pairs of jaws of the end effector, unclips it from the passive clip adjacent to the cell consumable 250, and pulls it over the bottom edge 300b of the tube connection section 300, and clips it into the first pair of cell consumable tube clips 500b-1, 500b-1′ in the second plurality of tube clips 500b.

As will be described later in relation to FIGS. 8A and 8B, the tube clips 500 are active tube clips 500 which engage and retain the tube 155-1.

In FIG. 5C, at step (1) the robotic device 5 engages the first cartridge consumable tube 155-1 (e.g., with the first pair of jaws of the end effector) and pulls it through the first pair of cartridge consumable tube clips 500a-1, 500a-1′. At step (2), the robotic device 5 continues pulling the tube 155-1 past the lower cell consumable tube clip 500b-1′ of the first pair of cell consumable tube clips 500b-1, 500b-1′. At step (3) the robotic device 5 engages the first cell consumable tube 255-2 below the lower cell consumable tube clip 500b-1′ (e.g., with the second pair of jaws of the end effector, while still retaining the first cartridge consumable tube 155-1 with the first pair of jaws of the end effector). Since both tubes 155-1, 255-1 are retained in a respective pair of jaws of the end effector, one or more of the tube clips, such as the lower cell consumable tube clip 500b-1′ may be opened so that the tubes 155-1, 255-1 may be pulled away from a surface of the tube connection section 300 by the robotic device 5.

Subsequently, the robotic device 5 may perform a tube weld between the first cartridge consumable tube 155-1 and the first cell consumable tube 255-1 thereby forming a single joined tube 355-1 between the first cartridge consumable 150-1 and the cell consumable 250. Once the weld is complete, at step (4) the robotic device 5 may move the joined tube 355-1 back towards the tube connection section 300 for the joined tube 355-1 to be re-engaged by one or more of the tube clips (i.e., the ones that were previously released before welding). The first and second pair of jaws of the end effector may now be opened to release the joined tube 355-1. Preferably, the end effector may pull the joined tube 355-1 upwards to remove any slack present between the first pair of cell consumable tube clips 500b-1, 500b-1′. In this way, the joined tube 355-1 may be engaged between the first pair of cell consumable tube clips 500b-1, 500b-1′ by a peristaltic pump (on the end effector), so that fluid may be pumped between the first cartridge consumable 150-1 and the cell consumable 250.

The formation of a subsequent connection will now be described in relation to FIG. 5D. At step (1), the tube sealer of the robotic device 5 seals the joined tube 355-1 between the first pair of cartridge consumable tube clips 500a-1, 500a-1′, preferably at the top of the gap therebetween. As a result, the joined tube 355-1 is split into a first cartridge consumable tube 155-1 and a first cell consumable tube 255-1. At step (2), the robotic device 5 engages a second cartridge consumable tube 155-2 (e.g., with the first pair of jaws of the end effector) and pulls it through the second pair of cartridge consumable tube clips 500a-1, 500a-2′. At step (3), the robotic device 5 continues to pull the tube 155-2 through the lower cartridge consumable tube clip 500a-2′ so that there is sufficient tubing below the tube clip 500a-2′ for the second cartridge consumable tube 155-2 to reach the first cell consumable tube 255-1 retained in the first pair of cartridge consumable tube clips 500a-1, 500a-1′. At step (4) the robotic device 5 engages the first cell consumable tube 255-1 (e.g. with the second pair of jaws of the end effector, while still retaining the second cartridge consumable tube 15-2 with the first pair of jaws of the end effector). Since both of the tubes 155-2, 255-1 are retained in a respective pair of jaws of the end effector, one or more of the tube clips, such as the lower first cartridge consumable tube clip 500a-1′ may be opened so that the tubes 155-2, 255-1 may be pulled away from the surface of the tube connection section 300 by the robotic device 5.

At step (5), the robotic device 5 may pull the tubes 155-2, 255-1 down so that they are both held below the lower first cartridge consumable tube clip 500a-1′. Subsequently, the robotic device 5 may perform a tube weld between the second cartridge consumable tube 155-2 and the first cell consumable tube 255-1 thereby forming a single joined tube 355-2 between the second cartridge consumable 150-2 and the cell consumable 250. The robotic device 5 may move the joined tube 255-2 back towards the tube connection section 300 for the joined tube 355-2 to be re-engaged by one or more of the tube clips (i.e., the ones that were previously released before welding). The first and second pairs of jaws of the end effector may now be opened to release the joined tube 355-2. As previously described, the robotic device 5 may perform further manipulations of the tubes to remove excess slack.

While the above steps have been described in relation to initially connecting the first cartridge consumable tube 155-1 to the first cell consumable tube 255-1, and subsequently connecting the second cartridge consumable tube 155-2 to the first cell consumable tube 255-1, it will be appreciated that the same steps apply to making connections between any of the other tubes 155, 255 connected to any of the consumables 150, 250.

### Alternative Fluid Connection Systems

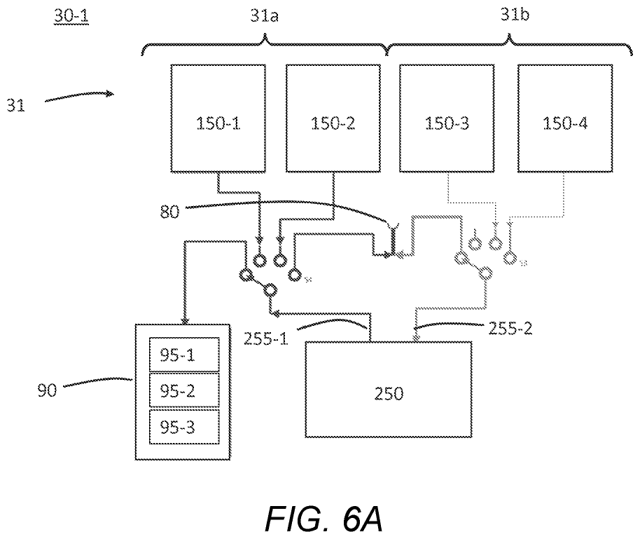

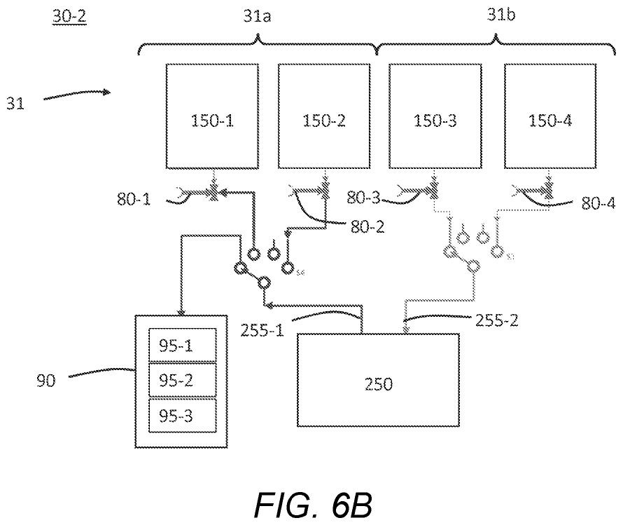

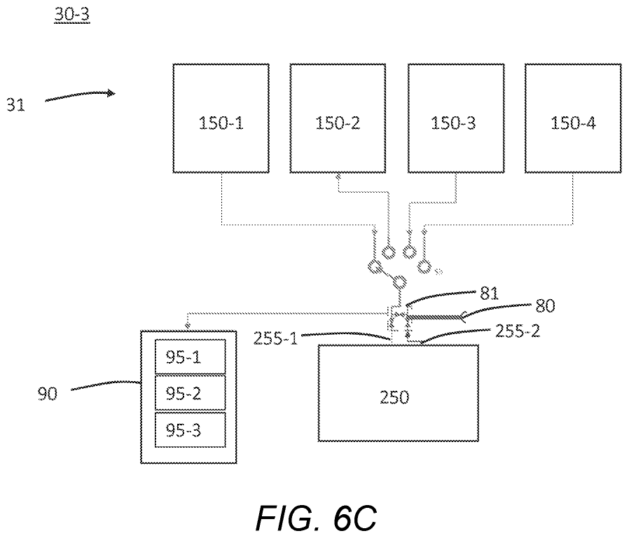

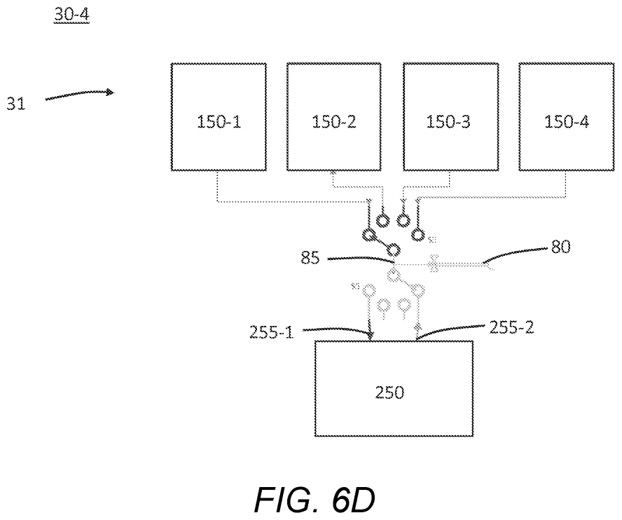

While the fluid connection system 30 shown in FIGS. 4A and 4B is preferred, a number of alternative fluid connection systems will now be described in relation to FIGS. 6A to 6D. Except where specified, these alternative systems may share a number of components with the preferred system already described; therefore, corresponding features may preferably be identified using corresponding reference numerals to avoid the need to describe said common features in detail for each and every alternative.

FIG. 6A shows a first alternative fluid connection system 30-1 (which may be known as the “above the line, weld on” arrangement). This system 30-1 is the same as the preferred fluid connection system 30, except that the sampling device 400 is not present, and instead an air supply 80 (such as a filter) is provided at the same location. The air supply 80 may also be used to purge tubes in a similar manner to as already described, but is not configured to collect fluid samples from the cell consumable 250. In order to collect samples, the fluid connection system 30-1 also includes a sampling unit 90 corresponding to the sampling unit 90 described in relation to FIG. 3, where the sampling unit 90 has a plurality of sample containers 95. Accordingly, while having a device 400 that performs both sampling and purging, the first alternative fluid connection system 30-1 demonstrates that this is not an absolute requirement. This system 30-1 is relatively simple to achieve; however, it is difficult to purge areas upstream of the welds, may require a greater number of welds in order to perform both sampling and purging, and requires installation of an additional consumable by the user.

FIG. 6B shows a second alternative fluid connection system 30-2 (which may be known as the “integrated into the consumable” arrangement). This system 30-2 is the same as the first alternative fluid connection system 30-1 except that instead of having an air supply 80 (in the same location as the device 400 in the preferred system), each of the cartridge consumables 150-1, 150-2, 150-3, 150-4 has a corresponding air supply 80-1, 80-2, 80-3, 80-4. For example, an air filter or an air containing bag may be pre-welded onto (e.g., each of) the cartridge consumables 150 with valving. The valving may comprise a 3-way valve or stopcock, switching between the air supply 80 and the cell consumable 250. Alternatively, the valving may comprise two pinch valves or a pinch valve and an inline check valve. The valving may be fully integrated into each of the cartridge consumables 150 and externally actuated (such as by a robotic device and/or an external solenoid). Alternatively, the function of the valving may be split between the cartridge consumable 150 and the bioprocessing system (i.e. the cartridge consumable 150 contains tubing between the air supply 80 (e.g., a filter) and the cartridge consumable 150 that may be installed onto valve seats in the bioprocessing system such as on the bioprocessing apparatus 1). In this way, purging can be achieved by actuating the valving and pumping air from one of the air supplies 80 through the connected tube to return the fluid to the cell consumable 250. Advantageously, fluid can be purged from any location in the connected tubes (not just downstream of the weld), and no connections or welds are required to form a weld. This allows simple “plug and play” operation for an operator. Further advantageously, this fluid connection system 30-2 and corresponding method enables the line to be fully purged with air before any subsequent welds are performed. This may reduce the risk of a weld failure since “dry” welds can be performed rather than wet welds (where there is fluid remaining in the tubes when a weld is performed). However, the cartridge consumables 150 are more complicated and need to be modified to include individual air supplies 80 (which increases the expense each consumable 150). Furthermore, there is a risk of multiple sterile filter failure, and therefore multiple filters may need to be pre-checked before use.

FIG. 6C shows a third alternative fluid connection system 30-3 (which may be known as the “switch and manifold” arrangement). In this system 30-3 there is only a single tube connection portion. In order to allow for connections to be made from any of the cartridge consumable 150 to the cell consumable 250, a valved manifold 81 is provided in order to enable switching between the cell consumable tubes 255-1, 255-2. The valved manifold 81 may be pre-attached to the cell consumable 250, and may include an air supply 80 such as an air filter. This enables the many-to-many connection between the cartridge consumables 150, the cell consumable tubes 255, and the sampling unit 90. Furthermore, the tubes required may be kept relatively short. In addition, the system 30-3 may be operated using only a single pump. However, the manifold 81 is a separate consumable that needs to be prepared and modified, thereby increasing the expense and complexity of the system 30-3.

FIG. 6D shows a fourth alternative fluid connection system 30-4 (which may be known as the “double switch partition” arrangement). In this system 30-4, the cell consumable tubes 255-1, 255-2 can be welded to a common intermediate tube 85, which is then welded to any of the cartridge consumable tubes 155. This also enables a many-to-many connection, and potentially may be operated using only a single pump. However, it requires the presence of the intermediate tube 85 and more welds may be required to form each connection. In order to perform purging, an air supply 80 may be attached to the intermediate tube 85. The air supply 80 may remain in place on the intermediate tube 85 even when other connections are made to ends of the intermediate tube. The air supply 80 may have a valve to enable pumping of air either upward toward the cartridge consumables 150 or downward to the cell consumable 250; in this way, purging can be performed in multiple directions before sealing. Advantageously, this system 30-4 is relatively simple to achieve, and enables purging in multiple directions. However, the switches on the air supply 80 may be complex, and the air supply is an additional consumable that needs to be installed by a user.

### Sampling Device

One process that is frequently performed in bioprocessing systems is sampling, where a small volume of fluid is extracted from the cell consumable 250 for testing, such as to monitor cell growth. The samples may have a volume from about 100 μl up to about 5 ml). For testing, the samples may need to be transported to a separate cell counter such as an “at-line” device. One way that this may be achieved is by welding a sampling consumable to the first tube 255-1 or the second tube 255-2 of the cell consumable 250. Subsequently fluid is pumped from the cell consumable 250 to the sampling consumable, and the sampling consumable is disconnected to facilitate testing (e.g., elsewhere in the bioprocessing system). However, since the tubes 255-1, 255-2 that connect between the cell consumable 250 and the sampling consumable may be long, a large volume of fluid may remain in the tubes 255-1, 255-2. A similar issue arises with long lengths of internal fluid routing within a rigid manifold; it will be appreciated that any of the features described herein in relation to flexible tubes may also apply to routing within a rigid fluid manifold. Especially if small samples (e.g. around 100 μl) need to be taken regularly from the cell consumable 250, this may lead to a large volume of fluid being wasted in the “dead volumes”. Furthermore, if fluid is left in the tubes 255-1, 255-2 for extended periods, the fluid can expire. If this expired fluid is subsequently mixed with other fluid within the bioprocessing system, it may contaminate the remaining fluid.

FIG. 7A shows a schematic diagram of a sampling device 400 which addresses the problem discussed above. The sampling device 400 has a fluid connection portion 410 which is configured to connect to a flexible tube 255 of a fluid-containing consumable (e.g., the cell consumable 250). While in this example, the cell consumable 250 is the bioreactor 250, it will be appreciated that the sampling device 400 may be used to sample from any cell-containing consumable within the bioprocessing system. The fluid connection portion 410 may comprise a fluid connection tube 412 that may be tube welded to other tubes in the bioprocessing system such as the tubes 255-1, 255-2 connected to the cell consumable 250. The sampling device 400 also comprises an air input portion 420 configured to allow sterile air to be supplied to the device 400. The air input portion 420 may be an air filter that filters air entering the device 400 from the surroundings; alternatively, the air input portion 420 may be a syringe or bag containing sterile air.

The fluid connection tube 412 leads to a first junction 413, which connects the fluid connection tube 412 to both a sampling portion 430 and a bypass portion 440. The first junction 413 splits one input into two outputs (or vice versa), and therefore may be provided by a y-connector, t-connector or similar.

The sampling portion 430 may comprise a sampling tube 432. The sampling tube 432 may have a first end 432a and a second end 432b, where the first end 432a of the sampling tube 432 is connected to the fluid connection portion 410 (e.g. via the junction 413). Preferably, the sampling portion 430 is connected to a sample container 434. In this way, when fluid is pumped into the fluid connection portion 410, the fluid fills the sample container 434. The sample container 434 may have a tapered shape, where fluid initially fills the narrow end of the taper before filling towards the wider end of the taper; in this way, the small volumes of fluid may be accurately measured when initially filling the narrow end of the taper, while still allowing the sample container 434 to receive larger samples of fluid. I.e. the sample container 434 provides proportional accuracy for both small samples and larger samples of fluid. Alternatively, the sampling tube 432 itself may have a length sufficient to retain the fluid sample. The sampling device 400 may include a level sensor in order to determine the volume of fluid collected in the sampling portion 430.

In this example, the second end 432b of the sampling tube 432 is connected to the air supply portion 420. In this way, when a fluid sample fills the sampling portion 430, the air displaced from the sampling portion 430 may leave the device 400 via the air input portion 420. Alternatively, the sample container 434 may comprise a bag or syringe that may expand and contract to accommodate the fluid pumped therein, thereby removing the need to connect the sampling portion 430 to the air input portion 420 (e.g. via the second end 432b of the sampling tube 432).

The bypass portion 440 comprises a bypass tube 442. The bypass tube 442 may have a first end 442a and a second end 442b. The first end 442a of the bypass tube 442 is connected to the fluid connection portion 410 (e.g. via the junction 413), and the second end 442b of the bypass tube 442 is connected the air input portion 420. Where both the sampling tube 432 and the bypass tube 442 are connected to the (same) air input portion 420, a second junction 423 may be present. For example, the air input portion 420 may comprise an air input tube 422, which is connected to both the second end 432b of the sampling tube 432 and the second end 442b of the bypass tube 442b by the second junction 423. The second junction 423 splits one input into two outputs (or vice versa), and therefore may be provide by a y-connector or t-connector. Alternatively, the sampling portion 430 may not be connected to the air input portion 420 or the sampling portion 430 and the bypass portion 440 may be connected to separate air input portions 420 (e.g. separate air filters or separate bags of sterile air).

The sampling device 400 also comprises a means for restricting fluid flow through the device 400, such that fluid being pumped into the fluid connection portion 410 passes only into the sampling portion 430, and air pumped out of the fluid connection portion 410 passes only through the bypass portion 440.

A method of collecting a sample may proceed generally as follows. First, the fluid connection portion 410 is connected to a flexible tube 255-1, 255-2 connected to a fluid-containing consumable (e.g., a cell consumable 250). This connection is preferably made by tube welding using a robotic device. Subsequently, fluid is pumped from the consumable 250 into the sampling portion 430 of the device 400 via the fluid connection portion 410. The pumping may be achieved using a peristaltic pump engaging the flexible tube 255-1, 255-2 (e.g. downstream of the sampling device 400). The means for restricting fluid flow ensures that the fluid entering the device 400 is diverted to the sampling portion 430 rather than entering the bypass tube 442. Once sufficient fluid is received in the sampling portion 430 (e.g. in the sample container 434), then the pumping direction is reversed. Instead of the fluid sample being removed from the sample container 434, the fluid restricting means ensures that air from the air input portion 420 is instead pumped out of the device 400 through the bypass portion 440. In other words, when pumping fluid remaining in the flexible tube 255-1, 255-2 back into the cell consumable 250, air is drawn from the air input portion 420 into the flexible tube 255-1, 255-2 via the bypass tube 442. This means that all of the fluid retained in the flexible tubes 255-1, 255-2 can be returned to the cell consumable 250, while still retaining the fluid sample in the device 400.

The means for restricting fluid flow through the device 400 may be provided by a first check valve 435 to allow fluid to flow only from the fluid connection portion 410 into the sampling portion 430. More specifically, the first check valve 435 is disposed along the sampling tube 432 and restricts flow of fluid to only moving from the first end 432a of the sampling tube 432 to the second end 432b of the sampling tube 432. The first check valve 435 only allows fluid to flow in one direction, which means that air from the air supply portion 420 does not purge the fluid sample from the sampling portion 430. The first check valve 435 is preferably located on the sampling tube 432 between the sample container 434 and the air input portion 420; in this way, the fluid sample does not need to pass through the first check valve 435.

The means for restricting fluid flow through the device 400 may be provided by a second check valve 445 to allow fluid to flow only from the air supply input portion 420 to the fluid connection portion 410 through the bypass tube 442. More specifically, the second check valve 445 is disposed along the bypass tube 442 and restricts flow of fluid (e.g. air) to only moving from the second end 442b of the bypass tube 442 to the first end 442a of the bypass tube 442. The second check valve 445 only allows fluid to flow in one direction, which means that fluid from the fluid connection portion 410 does not enter the bypass tube 442.

As an alternative to using either or both of the check valves 435, 445, the means for restricting fluid flow may be provided by a portion of pinchable tubing. For example, the sampling tube 432 and/or the bypass tube 442 may be formed from flexible tubing that may be pinched by an external pinch valve, during use. During pumping of fluid into the device 400, the bypass tube 442 may be pinched so that fluid is diverted into the sampling tube 432; during pumping of fluid out of the device 400, the sampling tube 432 may be pinched so that air is diverted from the air input portion 420 to the fluid connection portion 410 via the bypass tube 442.

The sampling portion 430 may comprise an extraction port (not shown in FIG. 7A), for removing the fluid sample from the sampling portion 430. For example, once fluid has been collected in the sample container 434, a syringe may be attached to the extraction port, and the fluid sample may be removed from the extraction port. The extraction port may comprise a needle free connector or a Luer lock capped port or a septum for use with a syringe needle.

The device 400 may comprise an external housing (not shown). The external housing may comprise a frame to hold the components of the device 400 in predetermined positions relative to each other; holding components of the device 400 such as the fluid connection portion 410, the air input portion 420 and the extraction port at predetermined positions facilitates consistent engagement by a robotic device. The external housing may have one or more engagement features to facilitate mounting of the device 400 within the bioprocessing system (e.g. so as to be retained within a tube clip), or to facilitate manipulation of the device 400 by a robotic device. Alternatively, or additionally, a multitude of sampling devices may be held within a single cartridge (e.g., a sampling cartridge), so that they can be easily loaded into the bioprocessing system by the user and then moved around the bioprocessing system by the robotic device 5. For example, the sampling cartridge may comprise a housing configured to retain one or more of the devices 400 (e.g., ten devices 400), which may be used one by one as needed within the bioprocessing system.

It will be appreciated that any of the components described above (e.g., the sampling tube 432) may be provided by several components joined together. FIG. 7B shows an exploded view of the device 400, where the individual parts are provided by the components shown in Table 1:

TABLE 1

| Numeral(s) | Description |
|---|---|
| 470 | Press-In Plug |
| 471 | Flexible tube, ⅛ inch ID and ¼ inch OD |
| 472 | Straight barbed connector |
| 473, 477, 479, 481 | PVC tubing with ⅛ inch ID and 3/16 inch OD. |
| 474, 476, 480 | Y connector with female Luer lock side arm |
| 475 | Soft PVC welded pillow with a volume of 4 ml |
| 477 | Male Luer cap |
| 478 | Check valve |
| 482 | Female Luer lock to barb connector |
| 483 | Transducer protector with female Luer lock inlet and |
| | male Luer lock outlet |
| 484 | Male Luer lock connector |
| 485 | PVC tubing with 0.118 inch ID and 0.161 inch OD |
| 486 | Check valve |

The soft PVC welded pillow 465 may be attached to the Y-connectors 474, 476 using cyanoacrylate adhesive bonding, for example.

The arrangement of components shown in FIG. 7B is purely exemplary and the shape of the device 400 can be modified. For example, FIG. 7C shows an alternative arrangement of the device 400 where the fluid connection portion 410 and the air input portion 420 are arranged adjacent to each other.

### Tube Clip

An example of a tube clip 500 will now be described in relation to FIGS. 8A and 8B. FIG. 8A shows the tube clip 500 when assembled, and FIG. 8B shows an exploded view of the tube clip 500 so that its internal components are visible. As shown, the components are generally assembled using a plurality of screws 590, though for clarity, not all of these screws 590 shown have been labelled.

The tube clip 500 comprises a main body 510, which may facilitate attachment of the tube clip 500 to other parts of a bioprocessing system such as to the tube connection section 300 described above.

The tube clip 500 has a tube seat 520 having a profile shaped to receive the tube at a predetermined position on the tube seat 520. In this example, the profile is substantially “C”-shaped. Alternatively, the profile may be generally “V”-shaped, rectangular, semi-circular or any other suitable shape for retaining receiving the tube at a predetermined position. The tube seat 520 is attached to a top surface 510a of the main body 510, such as by one or more screws.

The tube clip 500 also has a retaining element 530 including a retaining arm 531. By movement of the retaining element 530, the retaining arm 531 may be moved relative to the tube seat 520 between an open position for allowing the tube to be received by the tube seat 520, and a closed position for retaining the tube in the predetermined position on the tube seat 520. The retaining arm 531 may have a step (or stepped portion) so that different diameters of the tube may be used with the same tube clip 500. The retaining arm 531 may be hooked.

Preferably, the tube seat 520 comprises a first (upper) portion 522 and a second (lower) portion 524, where the upper and lower portions 522, 524 of the tube seat 520 are spaced apart parallel to an axis of the tube to provide a gap within which the retaining arm 531 is arranged to contact the tube. In this way, the retaining arm 531 applies a symmetrical force to the tube so that it is not bent during engagement.

The tube clip 500 also comprises an actuator 540 configured to move the retaining arm 531 between the open and closed positions. The actuator 540 is controllable to move the retaining arm 531 between a plurality of intermediate positions. This allows different diameters of tube to be correctly engaged (and/or pinched) by the tube clip 500, such as 4 mm tube, or 6 mm tube. Furthermore, in each of the intermediate positions the retaining arm 531 may apply a different force to the tube against the tube seat 520 whereby to control fluid flow through the tube. For example, the closed position may apply a force that fully pinches the tube shut (i.e., occludes the tube) to prevent any flow of fluid therethrough. An intermediate position may tightly retain the tube to restrict (but not prevent) flow of fluid therethrough. Another intermediate position may lightly retain the tube without substantially restricting flow of fluid therethrough. Preferably, the actuator 540 may move the retaining arm 531 through a continuous range of positions in order to apply continuous range of forces to a retained tube.

The actuator 540 is contained inside the main body 510. The actuator 540 connects to the retaining element 530 by a shaft 542 that extends out a top surface 510a of the main body 510. The actuator 540 is preferably a motor, that provides rotational motion of the shaft 542, thereby rotating the retaining arm 531 to move between the open and closed positions. The retaining element 530 may be biased by a spring 534 to oppose the force applied by the actuator 540; in this way, the retaining arm 531 is automatically moved to the closed position when the actuator 540 is disengaged. More specifically, the spring 534 pulls the retaining arm 531 to the closed position to engage (but not pinch) the tube. This provides a fail-safe against dropping the tube and may allow the actuator 540 to only need to be controllable to rotate in one direction.

In addition to the rotational motion of the retaining element 530, the force from the actuator 540 is transmitted to the retaining element 530 by a camming element 528 (referred to herein as a “pinch cam”) so that the retaining arm 531 also moves laterally when moving between the open and closed positions. The pinch cam 528 is mounted inside an opening 525 of the tube seat 520. A first bearing 526 is provided within this opening 525 to facilitate smooth movement of the pinch cam 528 relative to the tube seat 520. The pinch cam 528 has a lobed portion, such that during continued rotation of the retaining element 530 (e.g., to bring the retaining arm 531 in front of the tube seat 520) the retaining arm 531 subsequently moves laterally towards the tube seat 520 to compress the tube between the retaining arm 531 and the tube seat 520. The spring 534 also provides regulation of this lateral movement.

A cap 544 may be provided to attach the retaining element 530 and the tube seat 520 to the main body 510 via the shaft 542. In order to facilitate smooth movement of the retaining element 530 relative to the cap 544, an opening 536 is provided in the retaining element 530 into which a second bearing 536 may be provided.

The cam 528 is mounted directly to the actuator 540 and is supported by the first bearing 526. An upper part of the cam 528 fits into the second bearing 536 which is offset from the rotation centre of the actuator 540. As a result of the offset, the retaining element 530 is moved laterally during rotation of the cam 528. On an opposite face of the cam 528 from the lobed portion, there is an arm drive that has a vertically protruding tab which engages with the retaining element 530; in this way, rotation of the cam 528 also rotates the retaining element 530 and retaining arm 531.

The tube clip 500 may also include a tube guide 550. The tube guide 550 may comprise a second tube seat 551 which is longitudinally offset (i.e. offset along the axis of a received tube) from the first tube seat 520. The tube guide 550 may also include a front fin 552 arranged to extend adjacent to the tube along the longitudinal axis. In this way, the tube guide 550 helps to retain the tube along a predetermined axis, thereby improving consistent engagement of the retained tube by an external robotic device. Since the front fin 552 is thinner than the spacing of the jaws of the robotic device when they grip the tube, the front fin 552 may continue supporting the tube during the engagement process. The tube guide 550 may be attached to a front surface 510b of the main body 510 of the tube clip 500 by one or more screws 590 (only some labelled).

The tube clip 500 may also comprise at least one sensor to detect one or more of: the presence of a portion of tube in the tube clip 500, and/or whether a portion of tube retained within the tube clip 500 contains fluid. For example, the at least one sensor may be a bubble sensor 560 arranged on the front surface 510b of the main body 510 of the tube clip, such that the longitudinal axis of a retained tube passes through the bubble sensor 560. The bubble sensor 560 is configured to detect whether the tube is wet or dry.

The at least one sensor may include a force sensor (not shown) to sense the force (e.g. torque) being applied to the tube by the retaining arm, thereby indicating whether a tube is present in the tube clip 500. The combination of the bubble sensor 560 and the force sensor allows the tube clip 500 to determine whether a tube is present, and whether the tube is wet or dry. The force sensor may be connected in a control loop to the actuator 540, thereby allowing a precise force to be applied to the tube. This is particularly advantageous where different sizes of tube may be used within the tube clip 500, since the actuator 540 can pinch to a predetermined consistent force (regardless of the tube size), which enables consistent engagement.

The tube clip 500 may comprise a processor 580, which is housed inside the main body 510. The processor 580 may receive external instructions, such as instructions to operate the actuator 540 to move the retaining arm 531 between the open position, the closed position and the plurality of intermediate positions. The processor 580 may receive measurements from the at least one tube sensor. These measurements may be recorded by the processor and/or transmitted externally. As mentioned above, the force sensor may be connected in a control (feedback) loop with the actuator 540, so that a precise force may be applied to the tube.

### Alternative Sampling and Purge Devices

A number of alternative sampling/purge devices and methods will now be briefly described.

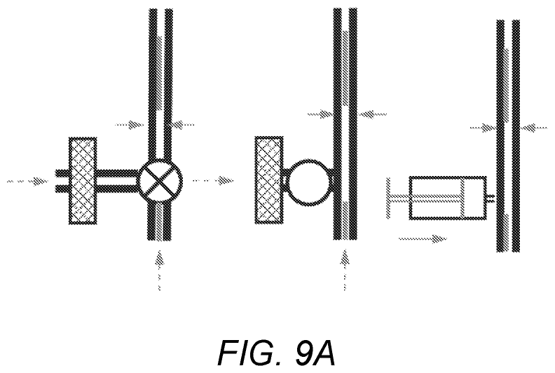

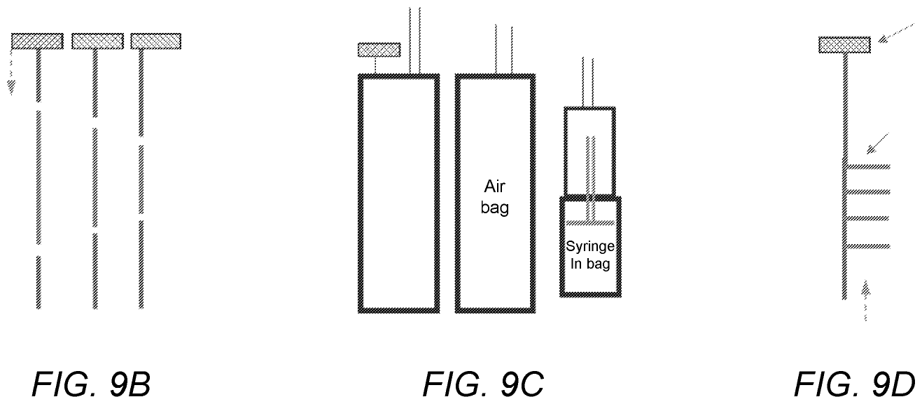

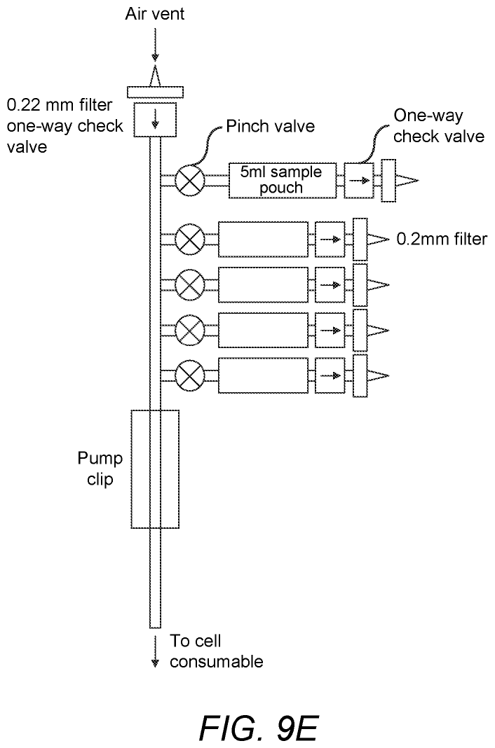

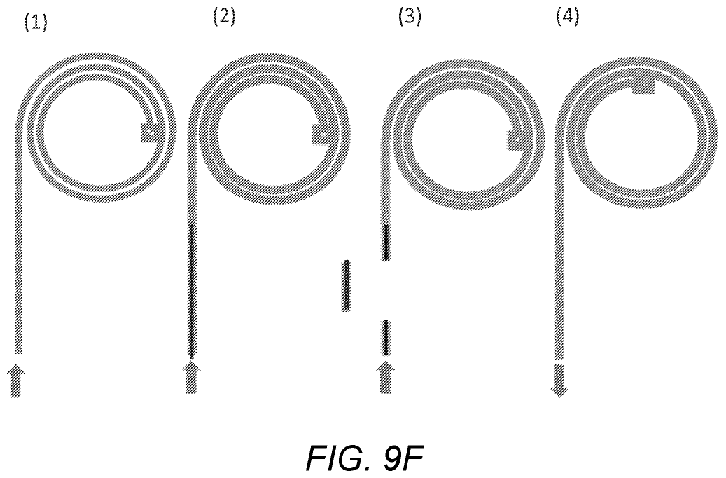

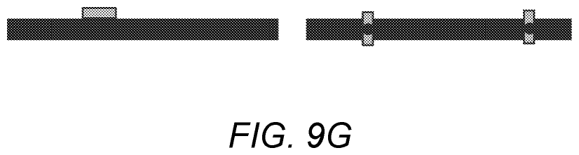

FIG. 9A shows a first way to extract a sample from the cell consumable, known as the “upstream” option. Here, the air source is inbuilt into the line, and may be used to generate “slugs” of the fluid in the tube. Then the tube may be sealed off and the sample may be removed. The steps may proceed as follows: (1) Install and weld on mechanically to the cell consumable (2) Install and weld on a sampling device onto tube (or additional length of tubing). (3) Pump fluid until there is a volume of fluid to be sampled past the valve, and then actuate valve and continue pumping to pump air into the line and pump the “slug” into the sampling device. (4) Seal off the sample. (5) Repeat steps (2)-(4) to generate subsequent samples. This process is deterministic, keeps tube lengths relatively short, allows variable volume samples to be taken (slug length depends on amount of pumping), and there is flexibility in the choice of the output container.

FIG. 9B shows a second way to extract a sample from the cell consumable, known as the “post hoc” option. Here, an air source is repeatedly welded onto a new sample line after the sample is taken. The steps may proceed as follows: (1) Weld on sample with filter. (2) Pump sample to level. (3) Weld sample section out and replace with a new section. (4) allow air to fall back and purge lines. This process is generally simple. However, since the weld lines keep changing, the sampling can become difficult when lots of samples are taken. Furthermore, it is difficult to keep the lengths of tubing short, and it is difficult to precisely control the volumes of the samples. Likewise, in order to provide flexibility for output sample containers, welds would need to be predefined (with welds on two sides).

FIG. 9C shows a third way to extract a sample from the cell consumable, known as the “built into consumable” option. The steps may proceed as follows: (1) weld on the sample. (2) optionally, purge the tube. (3) pump into the sample container. (4) seal. This is relatively simple, where the only constraint is that the sample container is welded in advance. This option is deterministic, though may be difficult for sample extraction. This option keeps the tubes relatively short, and allows variable volumes to be extracted (though may be difficult for smaller samples). Flexibility of output sample container may also be difficult due to the presence of the air providing an air spring that resists fluid movement.

FIG. 9D, shows a fourth way to extract a sample from the cell consumable, known as the “top air line with expandable volume” option. Here, a continuous tube is provided leading to an air supply (air filter or expandable volume), with offshoots providing sealable, expandable volumes (e.g., a bag or syringe). The steps may proceed as follows: (1) Install and weld on the sampling apparatus. (2) unblock the offshoot to be sampled into. (3) pump into the selected offshoot (preferably, where the tube to the air supply is blocked). (4) seal of the selected offshoot and remove. While not particularly simple, this option is deterministic, keep tube lengths moderately short, allows variable volumes for fluid samples, and allows flexibility of the output container.

FIG. 9E shows a fifth way to extract a sample from the cell consumable, known as the “pinch valve manifold” option, which is a variation of the “top air line with expandable volume” option. Here, pinch valves are provided on the offshoots.

FIG. 9F shows a sixth way to extract a sample from the cell consumable, known as the “sampling line draw down” option, which is a variation of the “post hoc” option. Here, a length of tube comes preloaded on a spool with a filter. Since the length of tube can be long, it is possible to use the spool to take a large number of samples. The steps may proceed as follows: (1) connect end of the tube to the cell consumable. (2) pump a volume of fluid into the tube from the cell consumable. (3) seal the tube on either side of the volume of fluid, and remove the sealed portion of tube. (4) pump remaining fluid outside the (new) end of the tube.

FIG. 9G shows a further variation of the “sampling line draw down” option. Here, a chain of consumables may be provided with wider inner diameter (e.g., 5 mm inner diameter to provide 5 cm per 1 ml samples). The chain may have pre-built in ports at regular spaced intervals or in built check valves and connectors. While providing more structure than the option in FIG. 9F, this does increase the complexity of manufacturing.

While the foregoing is directed to exemplary embodiments of the present invention, it will be understood that the present invention is described herein purely by way of example, and modifications of detail can be made within the scope of the invention. Furthermore, one skilled in the art will understand that the present invention may not be limited by the embodiments disclosed herein, or to any details shown in the accompanying figures that are not described in detail herein or defined in the claims. Indeed, such superfluous features may be removed from the figures without prejudice to the present invention.

Moreover, other and further embodiments of the invention will be apparent to those skilled in the art from consideration of the specification, and may be devised without departing from the basic scope thereof, which is determined by the claims that follow.

1. A device for collecting a fluid sample from a fluid-containing consumable in a bioprocessing system, the consumable having a tube fluidly attached thereto, the device comprising:
a fluid connection portion configured to be connected to the tube of a fluid-containing consumable, and to enable pumping of fluid or air through the device; and
an air input portion configured to allow sterile air to be supplied to the device;
a sampling portion connected to the fluid connection portion, wherein during pumping into the device, the sampling portion is configured to receive a fluid sample from the consumable via the fluid connection portion;
a bypass tube having a first end connected to the fluid connection portion and a second end connected to the air input portion, wherein the bypass tube is configured to receive air supplied from the air input portion and direct the air out of the device via the fluid connection portion without passing through the sampling portion, whereby to purge fluid from the tube connected to the consumable; and
means for restricting fluid flow through each of the sampling portion and bypass tube, such that fluid being pumped into the fluid connection portion passes only into the sampling portion and air pumped out of the fluid connection portion passes only through the bypass tube.

2. The device of claim 1, further comprising a sampling tube having a first end connected to the fluid connection portion, and a second end connected to the air input portion, wherein the sampling portion is provided between the first end and the second end of the sampling tube.

3. The device of claim 2, wherein the sampling portion is provided by a sample container disposed between the first end and the second end of the sampling tube.

4. The device of claim 3, wherein the sample container has a tapered shape.

5. The device of claim 1, wherein the sampling portion comprises an extraction port for removing the fluid sample from the sampling portion.

6. The device of claim 1, wherein the means for restricting fluid flow through the device comprises a check valve to allow fluid to flow from the fluid connection portion into the sampling portion.

7. The device of claim 6, further comprising a sampling tube having a first end connected to the fluid connection portion, and a second end connected to the air input portion, wherein the sampling portion is provided between the first end and the second end of the sampling tube, wherein the check valve is located on the sampling tube between the sampling portion and the air input portion.

8. The device of claim 1, wherein the means for restricting fluid flow through the device comprises a check valve to allow air to flow from the air input portion to the fluid connection portion through the bypass tube.

9. The device of claim 1, wherein the fluid connection portion comprises a length of flexible tube, thereby enabling connection of the device to the tube of the consumable by tube welding.

10. The device of claim 1, wherein the air input portion is an air filter configured to supply sterilised air from the surroundings.

11. The device of claim 1, wherein the air input portion comprises a container of sterile air.

12. The device of claim 1, wherein the air input portion is a single air input portion connected to both the sampling portion and the bypass tube.

13. The device of claim 1, further comprising an external housing.

14. A sampling cartridge comprising a housing, the housing configured to retain one or more of the devices of claim 1.

15. A method of collecting a fluid sample using the device of claim 1, comprising:
connecting the fluid connection portion to a tube connected to a consumable;
pumping fluid from the consumable into the sampling portion of the device via the fluid connection portion; and
pumping fluid remaining in the tube back into the consumable, thereby drawing air from the air input portion into the tube via the bypass tube.

16.-37. (canceled)
